(19) United States
(12) Patent Application Publication
(10) Pub. No.: US 2026/0176264 A1
(43) Pub. Date: Jun. 25, 2026

(54) METHODS OF SYNTHESIS FOR CUCURBITURIL COMPOUNDS

(71) Applicant: Clear Scientific, Inc., Cambridge, MA (US)

(72) Inventors: Xinhua Li, Newton, MA (US); Daniel Wallach, Melrose, MA (US); Bernard Wang, Guangzhou City (CN); Runsheng Mai, Guangzhou City (CN); Piercen Oliver, Malden, MA (US); Patrick Reust, Medford, MA (US); Rachel Korte, Cambridge, MA (US)

(21) Appl. No.: 19/122,324

(22) PCT Filed: Oct. 20, 2023
(86) PCT No.: PCT/US23/77486

§ 371 Date: Apr. 18, 2025

Related U.S. Application Data
(60) Provisional application No. 63/380,318, filed on Oct. 20, 2022.

(51) Int. Cl. C07D 471/04; C07D 413/14; C07D 417/14

(52) U.S. Cl. CPC C07D 471/04; C07D 413/14; C07D 417/14

(57) ABSTRACT

Disclosure herein relates to, inter alia, inventive cucurbituril compounds and novel methods of preparing thereof using a glycoluril compound having the formula (II), a glycoluril compound having the formula (II′), a glycoluril bis-ether of formula (III), a glycoluril dimer of formula (IV), a glycoluril tetramer compound of formula (V), and Sodium 4,4′-(1,4-phenylenebis(oxy))bis(butane-1-sulfonate, which result in significantly improved purity and yield of the intermediates and of the final products.

This invention was made with government support under grant no. U01DA053054 awarded by the National Institute of Health. The government has certain rights in the invention.

### FIELD

The disclosures herein relate to, inter alia, cucurbituril compounds and methods of preparing the cucurbituril compounds.

### BACKGROUND

Cucurbiturils are a class of macrocyclic compounds based on oligomers of glycoluril, its analogues and derivatives. Cucurbiturils can be used to form complexes with other molecules and are useful as sequestering agents. This property makes cucurbiturils an attractive candidate for the entrapment and removal of chemical agents, such as toxins and pollutants.

Cucurbiturils represent a promising technology for reversing methamphetamine intoxication and/or overdose. There is an urgent need for a safe rapidly acting reversal agent for methamphetamine. Methamphetamine (meth) is the fastest growing drug of abuse in the U.S., representing over 200,000 annual emergency room (ER) visits, with deaths quadrupling since 2015 (9356 deaths in 2017 alone), yet no current therapeutics are available for treating meth intoxication. Meth induces excessive, rapid, and sustained (serum half-life t_{1/2}˜10 hr) stimulation of the sympathetic nervous system, responsible for a recognizable adrenergic toxidrome consisting of both behavioral (psychomotor agitation) and physiological (tachycardia, hypertension, mydriasis, and diaphoresis) components. Meth intoxication can cause cardiovascular and other complications; 20% of ER visits require hospital admission. Current standard of care for meth intoxication is to treat symptoms only. A reversal agent for meth that normalizes both the behavioral and physiological components would minimize complications, improve patient outcomes, and reduce mortality.

### SUMMARY

Provided herein, inter alia, are methods of preparing cucurbituril compounds, which may be suitable for large scale synthesis and which result in significantly improved purity and yield of the intermediates and of the final products.

In one aspect, the disclosure provides a method for the preparation of a compound having the formula I:

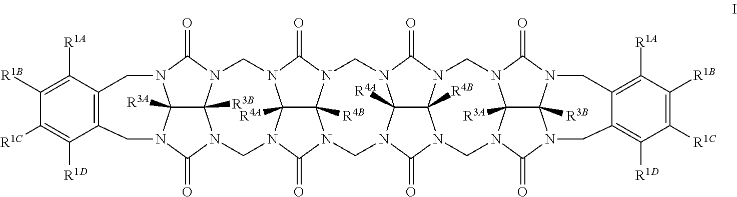

or a pharmaceutically acceptable salt thereof,
wherein:
   each R^{1A} and R^{1D} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹;
   each R^{1B} and R^{1C} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹; or
   additionally or alternatively, two R^{1A}, R^{1B}, R^{1C} and R^{1D} attached on the same phenyl ring at adjacent positions, together with atoms to which they are attached, are joined to form a fused C₆-C₁₂ aryl, 5 to 12 membered heteroaryl, or 5 to 7 membered heterocycle, which are optionally substituted with 1 to 3 substituents independently selected halogen, —OH, —NH₂, substituted or unsubstituted C₁-C₆ alkyl, or substituted or unsubstituted 2 to 6 membered heteroalkyl;
   each R^{3A} and R^{3B} is independently selected from hydrogen, halogen, —OH, C₁-C₆ alkyl, phenyl, substituted phenyl and 2 to 6 membered heteroalkyl;
   each R^{4A} and R^{4B} is independently selected from hydrogen, halogen, —OH, C₁-C₆ alkyl, phenyl, substituted phenyl and 2 to 6 membered heteroalkyl;
   each n1 is independently selected from 0 to 5;
   each v1 is independently selected from 2 or 3; and
   each X¹ is independently selected from selected from H, —OH, C₁-C₆ alkyl, alkali metal cation, and quaternary ammonium cation.

The method for preparing the compound of formula I includes one or more of the steps of:
(i) contacting a compound having the formula II:

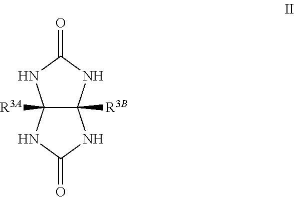

and paraformaldehyde in the presence of acid to provide a compound having the formula III:

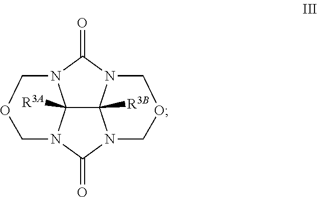

(ii) contacting a compound having the formula II′:

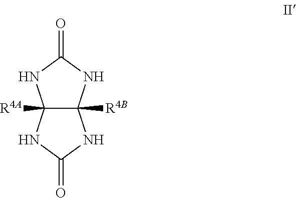

with paraformaldehyde and acid, wherein the paraformaldehyde is added to the compound of formula II′ over a period of at least about 1 hour, and wherein the reaction is maintained at a temperature above 55° C., to provide a compound having the formula IV:

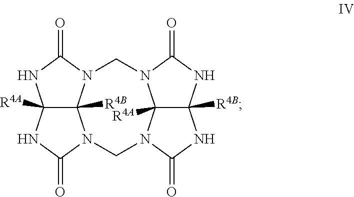

(iii) contacting a compound having the formula III:

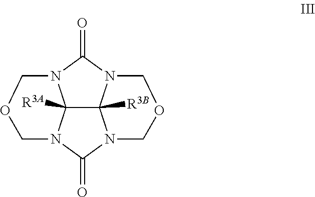

and a compound having the formula IV in the presence of (a) one or more acids having a pKa of less than about 1, and optionally (b) one or more polar aprotic solvents and/or additional acid solvents, to provide a compound having the formula V:

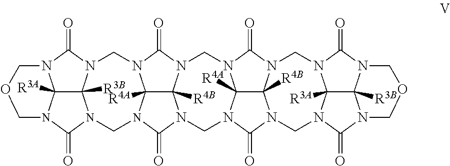

and isolating the compound of formula V;
(iv) contacting the compound having the formula V and a compound having the formula VI:

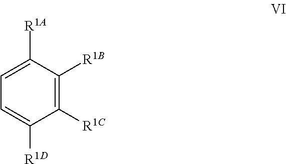

in the presence of a solvent comprising one or more acids having a pKa of less than about 1 and anhydride to provide the compound having the formula I.

In embodiments, the reaction of step (i) is maintained at a temperature above about 35° C.

In embodiments, in the reaction of step (ii) the paraformaldehyde is added in four or more portions separated by at least about 10 minutes.

In embodiments, the reaction of step (ii) is maintained at a temperature above 65° C.

In embodiments, the reaction mixture of step (ii) does not solidify during the reaction process.

In embodiments, the method further includes a step of isolating the compound of formula IV in step (ii), which includes contacting the reaction mixture with water, heating to a temperature of at least about 70° C., cooling to a temperature below about 30° C., and centrifuging or filtering the reaction mixture.

In embodiments, the reaction temperature of step (iii) is maintained at or above about 55° C.

In embodiments, the solvent of step (iii) includes an acid selected from the group consisting of trifluoroacetic acid, phosphoric acid, sulfuric acid, HCl, HBr, HClO₄, HNO₃, triflic acid, methane sulfonic acid, toluene sulfonic acid, Eaton's reagent, and combinations thereof.

In embodiments, the polar aprotic solvent of step (iii) includes dimethylformamide (DMF), dimethyl sulfoxide (DMSO), acetonitrile, N,N-dimethylacetamide (DMA), N-methyl-2-pyrrolidone (NMP), tetrahydrofuran (THF), dioxane, sulfolane, acetone, N,N′-dimethylpropyleneurea (DMPU), diglymes, hexamethylphosphoramide (HMPA), or combinations thereof.

In embodiments, the acid of step (iii) is trifluoroacetic acid (TFA), which may also be a solvent.

In embodiments, the method further includes a step of isolating the compound of formula V, which includes adding additional TFA to the reaction mixture after completion of the reaction; precipitating the compound of formula V; and filtering a mixture comprising the compound of formula V.

In embodiments, the step of precipitating the compound of formula V includes adding a countersolvent for the compound of formula V. The countersolvent may include methanol, ethanol, isopropyl alcohol, acetone, acetonitrile, tetrahydrofuran (THF), dioxane, brine, or combinations thereof.

In embodiments, in step (iii) or (iv), the one or more acids, respectively, having a pKa of less than about 1 includes trifluoroacetic acid, Eaton's reagent, methane sulfonic acid (MeSO₃H), toluene sulfonic acid (TsOH), triflic acid, phosphoric acid, sulfuric acid, camphorsulfonic acid (CSA), ethane sulfonic acid (EtSO₃H), HCl, HBr, HClO₄, HNO₃, or combinations thereof.

In embodiments, each R^{1A} and R^{1D} is —O—(CH₂)ₙ₁S(O)ᵥ₁X¹, —O—(CH₂)ₙ₁CO₂X¹, or —O—(CH₂)ₙ₁POᵥ₁X¹.

In embodiments, R^{1B} and R^{1C} are hydrogen.

In embodiments, X¹ is H, or alkali metal cation.

In embodiments, each R^{3A} and R^{3B} is independently C₁-C₃ alkyl.

In embodiments, R^{4A} and R^{4B} are hydrogen.

In another aspect, the disclosure provides a method for the preparation of a compound of Formula VI-A:

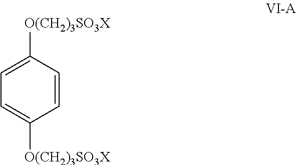

wherein:
   X is selected from H, an alkali metal cation, and a quaternary ammonium cation.

The method includes steps of:
(i) contacting a hydroquinone having the formula

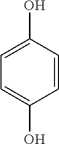

and a sultone having the formula

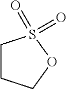

in the presence of about 2.0 to 2.5 equivalents of a base, and a solvent comprising one or more of water, polar aprotic solvents, and combinations thereof.

In embodiments, the base is present at about 2.2 to 2.4 equivalents.

In embodiments, the base may include an alkali metal hydroxide such as LiOH, NaOH and KOH, an alkali metal carbonate such as Na₂CO₃ or K₂CO₃. Alternatively or additionally, the base may include a non-nucleophilic organic base such as diisopropylethylamine (DIPEA), 1,8-diazabicyclo[5.4.0]undec-7-ene (DBU) and the like.

In embodiments, the sultone is added over a period of greater than about 2 hours.

In embodiments, the sultone is added in two or more portions separated by at least one hour.

In embodiments, the reaction mixture is cooled to less than about 10° C. prior to each addition of the sultone.

In embodiments, the method further includes a step of adding a base to the reaction in an amount equivalent to or greater than an amount of the sultone.

In embodiments, the base is added in two or more portions separated by at least one hour.

In another aspect, the disclosure provides a method for the preparation of a compound having the formula I-A

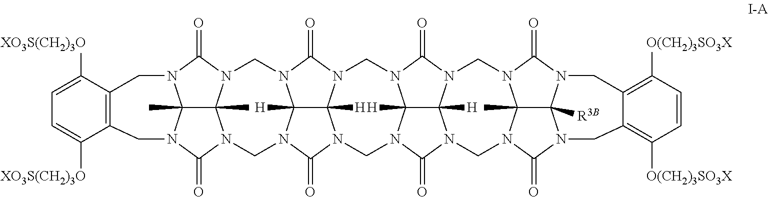

wherein
   X is selected from H, an alkali metal cation and a quaternary ammonium cation, and combinations thereof.

The method includes steps of:
(i) contacting a hydroquinone having the formula

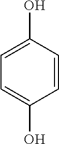

and a sultone having the formula

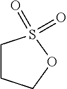

in the presence of about 2.0 to 2.5 equivalents of a base, and a solvent comprising one or more of water, polar aprotic solvents, and combinations thereof, to provide a compound having formula VI-A:

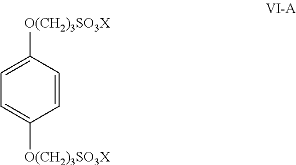

(ii) isolating the compound of formula VI-A; and
(iii) contacting the compound of formula VI-A and a compound having the formula V-A:

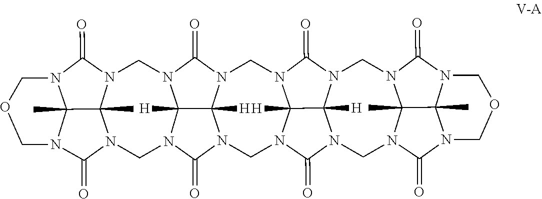

to provide the compound having the formula I-A.

In embodiments, the base of step (i) is present at about 2.2 to 2.4 equivalents.

In embodiments, the base of step (i) includes an alkali metal hydroxide.

In embodiments, the sultone of step (i) is added over a period of greater than about 2 hours.

In embodiments, the sultone is added in two or more portions separated by at least one hour.

In embodiments, the reaction mixture of step (i) is cooled to less than about 10° C. prior to each addition of the sultone.

In embodiments, the method further includes a step of adding a base to the reaction in an amount equivalent to or greater than an amount of the sultone.

In embodiments, the base is added in two or more portions separated by at least one hour.

Other aspects are disclosed infra.

### BRIEF DESCRIPTION OF THE FIGURES

FIG. 1 shows an exemplary synthesis of the compound CS-1103 according to an exemplary embodiment.

FIG. 2 shows an exemplary synthesis of the compound CS-1105 according to an exemplary embodiment.

### DETAILED DESCRIPTION

Provided herein, inter alia, are cucurbituril compounds and methods for manufacturing of cucurbiturils. The synthesis process in the methods described herein provide one or more of the following benefits: improved yield and purity for key intermediates and for the final product, place in-process controls to prevent the formation of sulfonate esters and other impurities, and improve the purification process allowing the key intermediate steps to be performed at scale.

### Definitions

The abbreviations used herein have their conventional meaning within the chemical and biological arts. The chemical structures and formulae set forth herein are constructed according to the standard rules of chemical valency known in the chemical arts.

The term “alkyl” refers to the radical of saturated aliphatic groups, including straight-chain alkyl groups and branched-chain alkyl groups. The alkyl may include a designated number of carbons (e.g., C₁-C₁₀ means one to ten carbons). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, and the like.

The term “alkenyl” refers to a linear or branched hydrocarbyl having at least one carbon-carbon double bond and including straight-chain and branched-chain alkenyl groups. Examples of alkenyl groups (e.g., “C₂-C₆ alkenyl”) includes vinyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, 4-methyl-3-pentenyl, and the like. When the compound of the present disclosure contains an alkenyl group, the compound may exist as the E-form, the Z-form, or any mixture thereof.

The term “alkynyl” refers to a linear or branched hydrocarbyl having at least one carbon-carbon triple bond and including straight-chain and branched-chain alkynyl groups. Examples of alkenyl groups (e.g., “C₂-C₆ alkynyl”) includes ethynyl, propynyl, and the like.

The term “cycloalkyl” refers to saturated, carbocyclic groups having from 3 to 9 carbons in the ring and including a monocyclic, bicyclic, or a multicyclic cycloalkyl ring system. Cycloalkyl groups include cyclopropyl, cyclobutyl, cyclopentyl and cyclohexyl. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non-adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Examples include, but are not limited to: —O—C₁-C₆ alkyl, —O—C₂-C₆ alkenyl, —O—C₂-C₆ alkynyl, —S—C₁-C₆ alkyl, —S—C₂-C₆ alkenyl, —S—C₂-C₆ alkynyl, —NH—C₁-C₆ alkyl, —NH—C₂-C₆ alkenyl, —NH—C₂-C₆ alkynyl, —N—(C₁-C₆ alkyl)₂, —S(O)—C₁-C₆ alkyl, —S(O)—C₂-C₆ alkenyl, —S(O)—C₂-C₆ alkynyl, —S(O)₂—C₁-C₆ alkyl, —S(O)₂—C₂-C₆ alkenyl, —S(O)₂—C₂-C₆ alkynyl, —C₁-C₆ alkyl-O—C₁-C₆ alkyl, —C₁-C₆ alkyl-S—C₁-C₆ alkyl, —C₁-C₆ alkyl-NH—C₁-C₆ alkyl, —C₁-C₆ alkyl-N—(C₁-C₆ alkyl)₂, —C₁-C₆ alkyl-S(O)—C₁-C₆ alkyl, —C₁-C₆ alkyl-S(O)₂—C₁-C₆ alkyl, and more particularly include, but are not limited to: —CH₂—O—CH₃, —CH₂—NH—CH₃, —CH₂—CH₂—N(CH₃)—CH₃, —CH₂—S—CH₃, —S(O)—CH₃, —CH₂—S(O)₂—CH₃, —Si(CH₃)₃, —O—CH₃, or —O—CH₂—CH₃. Up to two or three heteroatoms may be consecutive, such as, for example, —CH₂—NH—OCH₃ and —CH₂—O—Si(CH₃)₃. The term “heteroalkenyl,” by itself or in combination with another term, means, unless otherwise stated, a heteroalkyl including at least one carbon-carbon double bond. The term “heteroalkynyl,” by itself or in combination with another term, means, unless otherwise stated, a heteroalkyl including at least one carbon-carbon triple bond.

The term “cycloalkenyl” as used herein is a monocyclic, bicyclic, or a multicyclic cycloalkenyl ring system. In embodiments, monocyclic cycloalkenyl ring systems are cyclic hydrocarbon groups containing from 3 to 9 carbon atoms, where such groups are unsaturated (i.e., containing at least one annular carbon-carbon double bond), but not aromatic. Examples of monocyclic cycloalkenyl ring systems include cyclopentenyl and cyclohexenyl. In embodiments, bicyclic cycloalkenyl rings are bridged or fused bicyclic rings.

The term “heterocycle,” “heterocyclyl” or “heterocyclic” as used herein, means a monocyclic, bicyclic, or multicyclic heterocycle. The monocyclic heterocycle is a 3, 4, 5, 6, 7 or 8 membered ring containing at least one heteroatom independently selected from the group consisting of O, N, S, Si, and P where the ring is saturated or unsaturated, but not aromatic. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. Representative examples of bicyclic heterocycles include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. The heterocycle is connected to the parent molecular moiety through any carbon atom or any nitrogen atom contained within the monocyclic or bicyclic ring system.

The term “aryl” as used herein includes 5- and 6-membered single-ring aromatic groups that may include from zero to four heteroatoms, for example, benzene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles”, “heteroaromatics” or “heteroaryl”. The term “aryl” also includes 7- to 14-membered polycyclic ring systems having two or more cyclic rings in which two or more carbons are common to two adjoining rings (the rings are “fused rings”) wherein at least one of the rings is aromatic (including heteroaryl), e.g., the other cyclic rings can be fused cycloalkyls, cycloalkenyls, aryls, heteroaryl and/or heterocyclic groups. Single-ring heteroaryl groups may have from 1 to 3 ring heteroatoms and fused polycyclic heteroaryl groups may have from 1 to 5 ring heteroatoms, wherein the ring heteroatoms are selected from N, O and S.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

It will be understood that “substituted”, “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Exemplary substituents as used herein means a group selected from oxo, halogen, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), or heteroaryl (e.g., 5 to 10 membered heteroaryl, 5 to 9 membered heteroaryl, or 5 to 6 membered heteroaryl), and these alkyl, heteroalkyl, cycloalkyl, heterocycloalkyl, aryl, and heteroaryl may be optionally substituted with at least one substituents. For example, in the cucurbituril compounds disclosed herein, each alkyl, heteroalkyl, cycloalkyl, heterocycloalkyl, phenyl, heteroaryl and heterocycle may be optionally substituted with 1 to 4 substituents selected from the foregoing substituents.

The term “sultone” as used herein refers to a cyclic ester including a hydroxy sulfonic acid

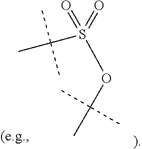

Exemplary sultone may be represented as

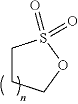

wherein, preferably, n is 1 to 3.

A quaternary ammonium cation as used herein is has the structure ⁺N(R)₄, wherein each R is independently selected from alkyl, cycloalkyl, aryl, aralkyl and heteroaryl, each of which may be optionally substituted. The quaternary ammonium cations, for example, may have the structure ⁺N(C₁₋₆ alkyl)₄, wherein each of the C₁₋₆ alkyl group boned to the nitrogen is independently selected.

Eaton's reagent is a phosphorus pentoxide solution in methanesulfonic acid. The wt % of the phosphorus pentoxide in the methane sulfonic acid may be from about 7% to about 15%, for example 7.5% or 10%.

The terms “a” or “an” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Certain compounds provided in this disclosure may exist in particular geometric or stereoisomeric forms. The disclosure contemplates all such compounds, including cis- and trans-isomers, R- and S-enantiomers, diastereomers, the racemic mixtures thereof, and other mixtures thereof, as falling within the scope of the invention. Additional asymmetric carbon atoms may be present in a substituent such as an alkyl group. All such isomers, as well as mixtures thereof, are included in this invention.

The term “pharmaceutically-acceptable salts” refers to the relatively non-toxic, inorganic and organic acid addition salts of compounds disclosed herein and inorganic and organic basic addition salts of the compounds disclosed herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like.

### Compounds

Provided herein are cucurbituril compounds that may be obtained or obtainable by one or more of the synthesis steps described herein.

In an aspect, a cucurbituril compound has a structure of formula I.

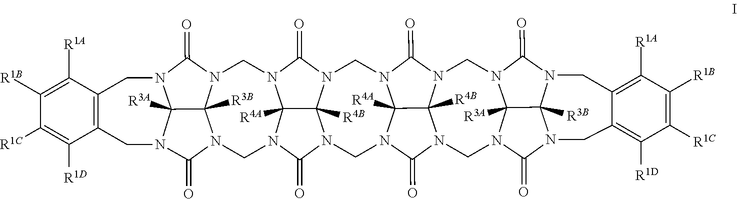

or a pharmaceutically acceptable salt thereof,
wherein:
   each R^{1A} and R^{1D} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹;
   each R^{1B} and R^{1C} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹; or
   additionally or alternatively, two R^{1A}, R^{1B}, R^{1C} and R^{1D} attached on the same phenyl ring at adjacent positions, together with atoms to which they are attached, are joined to form a fused C₆-C₁₂ aryl, 5 to 12 membered heteroaryl, or 5 to 7 membered heterocycle, which are optionally substituted with 1 to 3 substituents independently selected halogen, —OH, —NH₂, substituted or unsubstituted C₁-C₆ alkyl, or substituted or unsubstituted 2 to 6 membered heteroalkyl;
   each R^{3A} and R^{3B} is independently selected from hydrogen, halogen, —OH, C₁-C₆ alkyl, phenyl, substituted phenyl and 2 to 6 membered heteroalkyl;
   each R^{4A} and R^{4B} is independently selected from hydrogen, halogen, —OH, C₁-C₆ alkyl, phenyl, substituted phenyl and 2 to 6 membered heteroalkyl;
   each n1 is independently selected from 0 to 5;
   each v1 is independently selected from 2 or 3; and
      each X¹ is independently selected from selected from H, —OH, C₁-C₆ alkyl, alkali metal cation, and quaternary ammonium cation.

In embodiments, R^{1B} is hydrogen, halogen, —OH, C₁-C₆ alkyl, 2 to 6 membered heteroalkyl, C₃-C₆ cycloalkyl, 5 to 6 membered heterocycloalkyl, phenyl, or 5 to 6 membered heteroaryl. In embodiments, RB is hydrogen, halogen, —OH, or C₁-C₆ alkyl, and particularly RB is hydrogen or halogen.

In embodiments, R^{1C} is hydrogen, halogen, —OH, C₁-C₆ alkyl, 2 to 6 membered heteroalkyl, C₃-C₆ cycloalkyl, 5 to 6 membered heterocycloalkyl, phenyl, or 5 to 6 membered heteroaryl. In embodiments, R^{1C} is hydrogen, halogen, —OH or C₁-C₆ alkyl, and particularly, R^{1C} is hydrogen or halogen.

In embodiments, R^{1B} and R^{1C} are hydrogen.

Additionally or alternatively, R^{1A} and R^{1B}, attached on the same phenyl ring, together with atoms attached thereto, may be joined to form a C₆-C₁₂ aryl, or 5 to 12 membered heteroaryl. In embodiments, R^{1A} and R^{1B}, attached on the same phenyl ring, together with atoms attached thereto, join to form phenyl. In embodiments, R^{1A} and R^{1B} attached on the same phenyl ring, together with atoms attached thereto, join to form naphthyl. In embodiments, R^{1A} and R^{1B}, attached on the same phenyl ring, together with atoms attached thereto, join to form pyridyl.

In embodiments, R^{1B} and R^{1C}, attached on the same phenyl ring, together with atoms attached thereto, join to form C₆-C₁₂ aryl, or 5 to 12 membered heteroaryl. In embodiments, R^{1B} and R^{1C}, attached on the same phenyl ring, together with atoms attached thereto, join to form phenyl. In embodiments, R^{1B} and R^{1C}, attached on the same phenyl ring, together with atoms attached thereto, join to form naphthyl. In embodiments, R^{1B} and R^{1C}, attached on the same phenyl ring, together with atoms attached thereto, join to form pyridyl.

In embodiments, R^{1C} and R^{1D}, attached on the same phenyl ring, together with atoms attached thereto, join to form C₆-C₁₂ aryl, or 5 to 12 membered heteroaryl. In embodiments, R^{1C} and R^{1D}, attached on the same phenyl ring, together with atoms attached thereto, join to form phenyl. In embodiments, R^{1C} and R^{1D}, attached on the same phenyl ring, together with atoms attached thereto, join to form naphthyl. In embodiments, R^{1C} and R^{1D} attached on the same phenyl ring, together with atoms attached thereto, join to form pyridyl.

In embodiments, the C₆-C₁₂ aryl or 5 to 12 membered heteroaryl, which are formed by two of R^{1A}, R^{1B}, R^{1C} and R^{1D} attached on the same phenyl ring, may be substituted with one or more substituents, e.g., halogen, —OH, —NH₂, substituted or unsubstituted C₁-C₆ alkyl, or substituted or unsubstituted 2 to 6 membered heteroalkyl.

In embodiments, R^{3A} is hydrogen or substituted or unsubstituted C₁-C₃ alkyl. In embodiments, R^{3A} is C₁-C₃ alkyl, and particularly methyl.

In embodiments, R^{3B} is hydrogen or substituted or unsubstituted C₁-C₃ alkyl. In embodiments, R^{3B} is C₁-C₃ alkyl, and particularly methyl.

In embodiments, R^{3A} and R^{3B} are both hydrogen. Alternatively, R^{3A} and R^{3B} may both be methyl. In embodiments, one of R^{3A} and R^{3B} is hydrogen and the other is methyl.

In embodiments, R^{4A} is hydrogen or substituted or unsubstituted C₁-C₃ alkyl. In embodiments, R^{4A} is C₁-C₃ alkyl, and particularly methyl. In embodiments, R^{4A} is H.

In embodiments, R^{4B} is hydrogen or substituted or unsubstituted C₁-C₃ alkyl. In embodiments, R^{4B} is C₁-C₃ alkyl, and particularly methyl. In embodiments, R^{4B} is hydrogen.

In embodiments, each R^{4A} and R^{4B} is hydrogen. Alternatively, each R^{4A} and R^{4B} may be methyl. In embodiments, one of R^{4A} and R^{4B} is hydrogen and the other is methyl.

In embodiments, each R^{3A} and R^{3B} is independently C₁-C₃ alkyl and R^{4A} and R^{4B} are hydrogen, and particularly, each R^{3A} and R^{3B} are methyl and each R^{4A} and R^{4B} are hydrogen.

In another aspect, the cucurbituril compounds that may be obtained or obtainable by one or more of the synthesis steps described herein have the structure of formula (I-a):

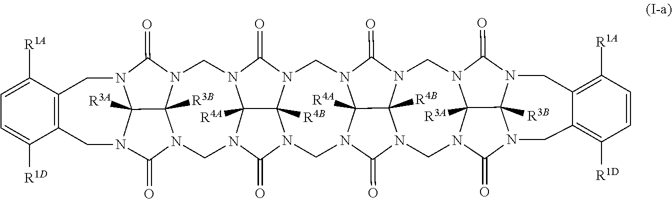

or a pharmaceutically acceptable salt thereof. R^{1A}, R^{1D}, R^{3A}, R^{3B}, R^{4A}, and R^{4B} are as described herein.

In embodiments, R^{3A} is hydrogen or substituted or unsubstituted C₁-C₃ alkyl. In embodiments, R^{3A} is C₁-C₃ alkyl, and particularly methyl.

In embodiments, R^{3B} is hydrogen or substituted or unsubstituted C₁-C₃ alkyl. In embodiments, R^{3B} is C₁-C₃ alkyl, and particularly methyl.

In embodiments, R^{3A} and R^{3B} are both hydrogen. Alternatively, R^{3A} and R^{3B} may both be methyl. In embodiments, one of R^{3A} and R^{3B} is hydrogen and the other is methyl.

In embodiments, R^{4A} is hydrogen or substituted or unsubstituted C₁-C₃ alkyl. In embodiments, R^{4A} is C₁-C₃ alkyl, and particularly methyl. In embodiments, R^{4A} is H.

In embodiments, R^{4B} is hydrogen or substituted or unsubstituted C₁-C₃ alkyl. In embodiments, R^{4B} is C₁-C₃ alkyl, and particularly methyl. In embodiments, R^{4B} is hydrogen.

In embodiments, each R^{4A} and R^{4B} is hydrogen. Alternatively, each R^{4A} and R^{4B} may be methyl. In embodiments, one of R^{4A} and R^{4B} is hydrogen and the other is methyl.

In embodiments, each R^{3A} and R^{3B} is independently C₁-C₃ alkyl and R^{4A} and R^{4B} are hydrogen, and particularly, each R^{3A} and R^{3B} are methyl and each R^{4A} and R^{4B} are hydrogen.

In embodiments, each R^{1A} and R^{1D} is neutral. In embodiments, each R^{1A} and R^{1D} is in an ionic salt form.

In embodiments, R^{1A} is —O—(CH₂)ₙ₁S(O)ᵥ₁X¹. In embodiments, R^{1A} is —O—(CH₂)ₙ₁CO₂X¹. In embodiments, R^{1A} is —O—(CH₂)ₙ₁POᵥ₁X¹. In embodiments, each n1 is 0. In embodiments, each n1 is 1. In embodiments, each n1 is 2. In embodiments, each n1 is 3. In embodiments, each n1 is 4. In embodiments, each n1 is 5. In embodiments, each v1 is 2. In embodiments, each v1 is 3.

In embodiments, R^{1D} is —O—(CH₂)ₙ₁S(O)ᵥ₁X¹. In embodiments, R^{1D} is —O—(CH₂)ₙ₁CO₂X¹. In embodiments, R^{1D} is —O—(CH₂)ₙ₁POᵥ₁X¹. In embodiments, each n1 is 0. In embodiments, each n1 is 1. In embodiments, each n1 is 2. In embodiments, each n1 is 3. In embodiments, each n1 is 4. In embodiments, each n1 is 5. In embodiments, each v1 is 2. In embodiments, each v1 is 3.

In embodiments, R^{1A} and R^{1D} attached to the same phenyl ring may be same or different. In embodiments, R^{1A} and R^{1A} attached to the different phenyl rings may be same or different. In embodiments, R^{1D} and R^{1D} attached to the different phenyl rings may be same or different.

In embodiments, each R^{1A} and R^{1D} attached to the same phenyl ring is independently —O—(CH₂)ₙ₁S(O)ᵥ₁X¹. In embodiments, each R^{1A} and R^{1A} attached to the different phenyl rings is independently —O—(CH₂)ₙ₁S(O)ᵥ₁X¹. In embodiments, each R^{1D} and R^{1D} attached to the different phenyl rings is independently —O—(CH₂)ₙ₁S(O)ᵥ₁X¹.

In another aspect, the cucurbituril compounds that may be obtained or obtainable by one or more of the synthesis steps described herein have the structure of formula (I-b):

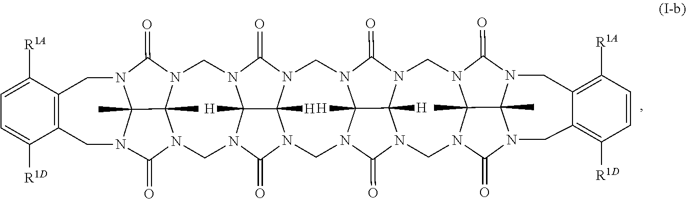

or a pharmaceutically acceptable salt thereof. R^{1A} and R^{1D} are as described herein.

In embodiments, each R^{1A} and R^{1D} is neutral. In embodiments, each R^{1A} and R^{1D} is in an ionic salt form.

In embodiments, R^{1A} is —O—(CH₂)ₙ₁S(O)ᵥ₁X¹. In embodiments, R^{1A} is —O—(CH₂)ₙ₁CO₂X¹. In embodiments, R^{1A} is —O—(CH₂)ₙ₁POᵥ₁X¹. In embodiments, each n1 is 0. In embodiments, each n1 is 1. In embodiments, each n1 is 2. In embodiments, each n1 is 3. In embodiments, each n1 is 4. In embodiments, each n1 is 5. In embodiments, each v1 is 2. In embodiments, each v1 is 3.

In embodiments, R^{1D} is —O—(CH₂)ₙ₁S(O)ᵥ₁X¹. In embodiments, R^{1D} is —O—(CH₂)ₙ₁CO₂X¹. In embodiments, R^{1D} is —O—(CH₂)ₙ₁POᵥ₁X¹. In embodiments, each n1 is 0. In embodiments, each n1 is 1. In embodiments, each n1 is 2. In embodiments, each n1 is 3. In embodiments, each n1 is 4. In embodiments, each n1 is 5. In embodiments, each v1 is 2. In embodiments, each v1 is 3.

In embodiments, R^{1A} and R^{1D} attached to the same phenyl ring may be same or different. In embodiments, R^{1A} and R^{1A} attached to the different phenyl rings may be same or different. In embodiments, RD and RD attached to the different phenyl rings may be same or different.

In embodiments, each R^{1A} and R^{1D} attached to the same phenyl ring is independently —O—(CH₂)ₙ₁S(O)ᵥ₁X¹. In embodiments, each R^{1A} attached to the different phenyl rings is independently —O—(CH₂)ₙ₁S(O)ᵥ₁X¹. In embodiments, each RD attached to the different phenyl rings is independently —O—(CH₂)ₙ₁S(O)ᵥ₁X¹.

In another aspect, the cucurbituril compounds that may be obtained or obtainable by one or more of the synthesis steps described herein have the structure of formula (I-c):

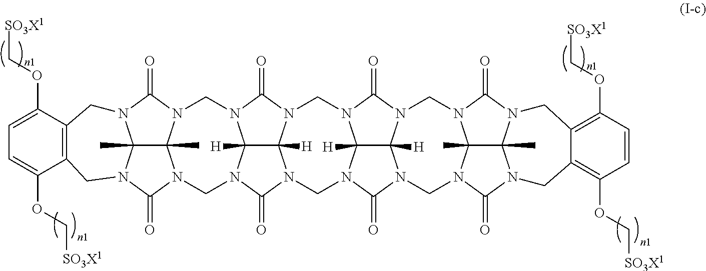

or a pharmaceutically acceptable salt thereof. X¹ and n1 are as described herein.

In embodiments, each X¹ are same or different. In embodiments, each X¹ is independently H, —OH, C₁-C₆ alkyl, alkali metal cation, or quaternary ammonium cation. In embodiments, each n1 is 0 to 5. In embodiments, each n1 is 1 to 5. In embodiments, each n1 is 2 to 5. In embodiments, each n1 is 3 to 5. In embodiments, each n1 is 4 or 5.

In another aspect, the cucurbituril compounds that may be obtained or obtainable by one or more of the synthesis steps described herein have the structure of formula (I-A),

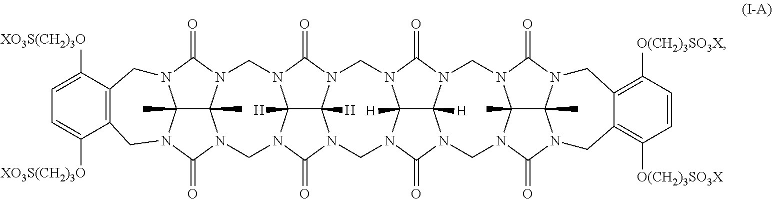

or a pharmaceutically acceptable salt thereof. Each X is independently H, alkali metal cation (e.g., Li⁺, Na⁺, K⁺, or Cs⁺), an ammonium cation, or combination thereof.

In another aspect, the cucurbituril compounds that may be obtained or obtainable by one or more of the synthesis steps described herein have the structure of formula (I-d),

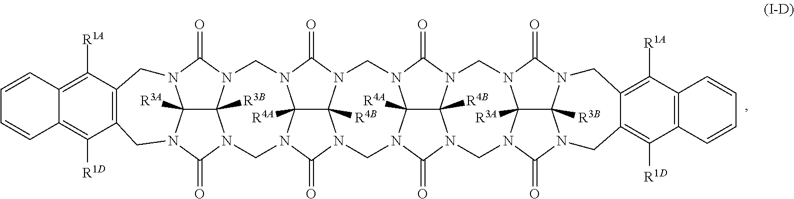

or a pharmaceutically acceptable salt thereof. R^{1A}, R^{1D}, R^{3A}, R^{3B}, R^{4A}, and R^{4B} are as described herein.

In embodiments, R^{3A} is hydrogen or substituted or unsubstituted C₁-C₃ alkyl. In embodiments, R^{3A} is C₁-C₃ alkyl, and particularly methyl.

In embodiments, R^{3B} is hydrogen or substituted or unsubstituted C₁-C₃ alkyl. In embodiments, R^{3B} is C₁-C₃ alkyl, and particularly methyl.

In embodiments, R^{3A} and R^{3B} are both hydrogen. Alternatively, R^{3A} and R^{3B} may both be methyl. In embodiments, one of R^{3A} and R^{3B} is hydrogen and the other is methyl.

In embodiments, R^{4A} is hydrogen or substituted or unsubstituted C₁-C₃ alkyl. In embodiments, R^{4A} is C₁-C₃ alkyl, and particularly methyl. In embodiments, R^{4A} is H.

In embodiments, R^{4B} is hydrogen or substituted or unsubstituted C₁-C₃ alkyl. In embodiments, R^{4B} is C₁-C₃ alkyl, and particularly methyl. In embodiments, R^{4B} is hydrogen.

In embodiments, each R^{4A} and R^{4B} is hydrogen. Alternatively, each R^{4A} and R^{4B} may be methyl. In embodiments, one of R^{4A} and R^{4B} is hydrogen and the other is methyl.

In embodiments, each R^{3A} and R^{3B} is independently C₁-C₃ alkyl and R^{4A} and R^{4B} are hydrogen, and particularly, each R^{3A} and R^{3B} are methyl and each R^{4A} and R^{4B} are hydrogen.

In embodiments, each R^{1A} and R^{1D} is neutral. In embodiments, each R^{1A} and R^{1D} is in an ionic salt form.

In embodiments, R^{1A} is —O—(CH₂)ₙ₁S(O)ᵥ₁X¹. In embodiments, R^{1A} is —O—(CH₂)ₙ₁CO₂X¹. In embodiments, R^{1A} is —O—(CH₂)ₙ₁POᵥ₁X¹. In embodiments, each n1 is 0. In embodiments, each n1 is 1. In embodiments, each n1 is 2. In embodiments, each n1 is 3. In embodiments, each n1 is 4. In embodiments, each n1 is 5. In embodiments, each v1 is 2. In embodiments, each v1 is 3.

In embodiments, R^{1D} is —O—(CH₂)ₙ₁S(O)ᵥ₁X¹. In embodiments, R^{1D} is —O—(CH₂)ₙ₁CO₂X¹. In embodiments, R^{1D} is —O—(CH₂)ₙ₁POᵥ₁X¹. In embodiments, each n1 is 0. In embodiments, each n1 is 1. In embodiments, each n1 is 2. In embodiments, each n1 is 3. In embodiments, each n1 is 4. In embodiments, each n1 is 5. In embodiments, each v1 is 2. In embodiments, each v1 is 3.

In embodiments, R^{1A} and R^{1D} attached to the same phenyl ring may be same or different. In embodiments, R^{1A} and R^{1A} attached to the different phenyl rings may be same or different. In embodiments, R^{1D} and R^{1D} attached to the different phenyl rings may be same or different.

In embodiments, each R^{1A} and R^{1D} attached to the same phenyl ring is independently —O—(CH₂)ₙ₁S(O)ᵥ₁X¹. In embodiments, each R^{1A} and R^{1A} attached to the different phenyl rings is independently —O—(CH₂)ₙ₁S(O)ᵥ₁X¹. In embodiments, each R^{1D} and R^{1D} attached to the different phenyl rings is independently —O—(CH₂)ₙ₁S(O)ᵥ₁X¹.

In another aspect, the cucurbituril compounds that may be obtained or obtainable by one or more of the synthesis steps described herein have the structure of formula (X),

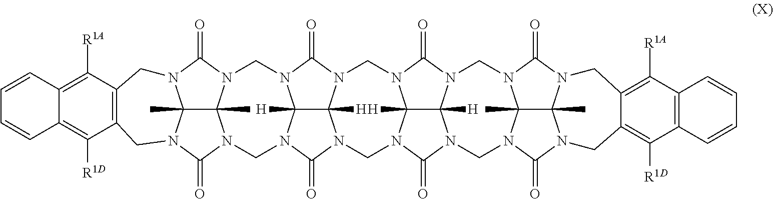

or a pharmaceutically acceptable salt thereof.

In embodiments, each R^{1A} and R^{1D} is neutral. In embodiments, each R^{1A} and R^{1D} is in an ionic salt form.

In embodiments, R^{1A} is —O—(CH₂)ₙ₁S(O)ᵥ₁X¹. In embodiments, R^{1A} is —O—(CH₂)ₙ₁CO₂X¹. In embodiments, R^{1A} is —O—(CH₂)ₙ₁POᵥ₁X¹. In embodiments, each n1 is 0. In embodiments, each n1 is 1. In embodiments, each n1 is 2. In embodiments, each n1 is 3. In embodiments, each n1 is 4. In embodiments, each n1 is 5. In embodiments, each v1 is 2. In embodiments, each v1 is 3.

In embodiments, R^{1D} is —O—(CH₂)ₙ₁S(O)ᵥ₁X¹. In embodiments, R^{1D} is —O—(CH₂)ₙ₁CO₂X¹. In embodiments, R^{1D} is —O—(CH₂)ₙ₁POᵥ₁X¹. In embodiments, each n1 is 0. In embodiments, each n1 is 1. In embodiments, each n1 is 2. In embodiments, each n1 is 3. In embodiments, each n1 is 4. In embodiments, each n1 is 5. In embodiments, each v1 is 2. In embodiments, each v1 is 3.

In embodiments, R^{1A} and R^{1D} attached to the same phenyl ring may be same or different. In embodiments, R^{1A} and R^{1A} attached to the different phenyl rings may be same or different. In embodiments, R^{1D} and R^{1D} attached to the different phenyl rings may be same or different.

In embodiments, each R^{1A} and R^{1D} attached to the same phenyl ring is independently —O—(CH₂)ₙ₁S(O)ᵥ₁X¹. In embodiments, each R^{1A} attached to the different phenyl rings is independently —O—(CH₂)ₙ₁S(O)ᵥ₁X¹. In embodiments, each RD attached to the different phenyl rings is independently —O—(CH₂)ₙ₁S(O)ᵥ₁X¹.

### Synthesis

Provided herein are methods of preparing the cucurbituril compounds described herein (i.e., compounds of formula I, Ia, Ib, Ic, I-A, I-D, X). The methods may include using one or more compounds having the formula (II), (II′), (III), (IV), (V), (VI), or (VI′). Also, provided herein are method of preparing a compound having the formula (I-A) and the methods may include using one or more compounds having the formula (II-A), (II′-A), (III-A), (IV-A), (V-A), or (VI-A).

In embodiments, the methods include the following Scheme 2.

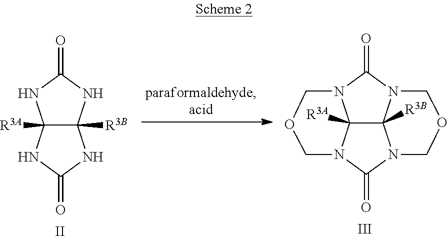

wherein R^{3A} and R^{3B} are as described herein. In embodiments, R^{3A} and R^{3B} are C₁₋₃ alkyl, or R^{3A} and R^{3B} are methyl.

The reaction according to Scheme 2 involves the treatment of a glycoluril compound having the formula II with paraformaldehyde in the presence of an acid to provide the glycoluril bis-ether of formula III. The paraformaldehyde is added in a molar excess relative to the glycoluril compound. The ratio of paraformaldehyde to the glycoluril compound may be from about 3 equivalents to about 8 equivalents, or from about 4 equivalents to about 6 equivalents.

Paraformaldehyde as used in Scheme 2 is a polyoxymethylene and is the polymerization product of formaldehyde, which may have a typical degree of polymerization of 8-100 units. The paraformaldehyde may be supplied as a solid or as solution, particularly as an aqueous solution.

The paraformaldehyde may be added to a mixture of the glycoluril compound, the acid and solvent in a reaction vessel. Prior to the addition of the paraformaldehyde to the reaction mixture, the temperature of the mixture is brought to at least 35° C. The temperature of the reaction mixture is maintained at 35° C. or above for the duration of the addition, after which the temperature may be maintained, or increased for example to 45° C. or above or to 50° C. or above, which increase may be temporary or maintained for the remainder of the reaction period, or even lowered from the addition temperature so long as the reaction temperature is maintained at or above about 35° C.

The reaction of Scheme 2 may be maintained at a temperature above about 35° C., or above about 36° C., or above about 37° C., or about 38° C., or above about 39° C., or above about 40° C., or above about 41° C., or above about 42° C., or above about 43° C., or about 44° C., or above about 45° C. The reaction of Scheme 2 may be maintained at a temperature in a range of about 35 to 55° C., or about 35 to 50° C., or about 35 to 40° C., or at about 35° C.

Heating the reaction mixture during the addition of the paraformaldehyde results in a significantly improved yield of the glycoluril bis-ether of formula III. In embodiments, the yield of the glycoluril bis-ether of formula III is greater than about 70%.

The acid used in the reaction mixture is a strong acid. In embodiments, the acid in the reaction of Scheme 2 may have a pKa of less than about 2.5, or a pKa of less than about 2.0, or a pKa of less than about 1.5, or a pKa of less than about 1, or a pKa of less than about 0.9, or a pKa of less than about 0.8, or a pKa of less than about 0.7, or a pKa of less than about 0.6, or a pKa of less than about 0.5. In embodiments, the acid in the reaction of Scheme 2 may have a pKa of less than about 0.9.

The acid in the reaction of Scheme 2 may include, but is not limited to, HF, HCl, HBr, HClO₄, HNO₃, H₂SO₄, H₃PO₄, sulfonic acids such as methane sulfonic acid, trifluoromethane sulfonic acid, ethane sulfonic acid, benzene sulfonic acid or toluene sulfonic acid, Eaton's reagent, trifluoroacetic acid, or combinations thereof. In embodiments, the acid in the reaction of Scheme 2 includes HF, HCl or HBr, and particularly, the acid is HCl. The molar concentration of the acid used in this reaction may range from about 5 to about 10 M, from about 6 to about 9 M, or from about 7 to about 9 M.

In embodiments, the methods include the following Scheme 3.

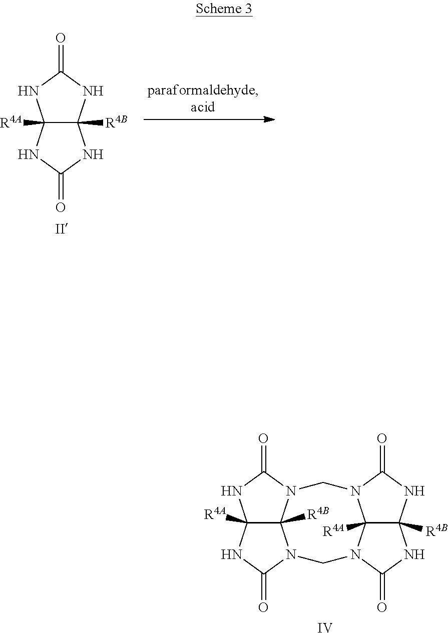

wherein R^{4A}, and R^{4B} are as described herein. In embodiments, R^{4A} and R^{4B} are hydrogen.

The reaction according to Scheme 3 involves the treatment of a glycoluril compound having the formula II′ with paraformaldehyde in the presence of an acid to provide the glycoluril dimer of formula IV. The solvent may be selected from water, alcohols such as methanol, ethanol and isopropanol, dimethylformamide (DMF), dimethyl sulfoxide (DMSO), acetonitrile, N,N-dimethylacetamide (DMA), N-methyl-2-pyrrolidone (NMP), tetrahydrofuran (THF), dioxane, sulfolane, acetone, N,N′-dimethylpropyleneurea (DMPU), diglymes, hexamethylphosphoramide (HMPA), or combinations thereof. In embodiments, the solvent is water, or water in combination with another solvent.

The paraformaldehyde is added in approximately the same or similar amount to the glycoluril compound. The ratio of paraformaldehyde to the glycoluril compound may be from about 0.5 equivalents to about 1.5 equivalents, or from about 0.8 equivalents to about 1.2 equivalents. The paraformaldehyde to the glycoluril compound may be in about 1.0 equivalent.

According to Scheme 3, the reaction mixture in Scheme 3 does not solidify during the reaction process. For example, the reaction mixture does nor form a gel during the reaction or after the reaction is quenched. Maintaining stoichiometry as the reaction proceeds may be important for selective production of the dimer of formula IV without occurrence of gelling of the reaction mixture. The paraformaldehyde as used in Scheme 3 may be supplied as a solid or as solution, particularly as an aqueous solution. The paraformaldehyde is added to a reaction vessel containing the compound of formula (II′) and acid over an extended period of time, e.g., added by dropwise or by several portions.

Maintaining careful stoichiometry as the reaction proceeds is important for selectively producing the glycoluril dimer of formula IV. Although the slow addition of the paraformaldehyde to the reaction mixture could be expected to disrupt reaction stoichiometry, the inventors have found that the yield and purity of the glycoluril dimer of formula IV was consistent or improved. Additionally, the slow addition of paraformaldehyde to the reaction mixture reduced or prevented the occurrence of gelling in the reaction mixture. The reaction proceeded more completely and with greatly improved workup, allowing the reaction to be used at a commercial scale.

Paraformaldehyde may be added to the compound of formula II′ in greater than 4 portions, for example in 4-10 portions, or in 5-8 portions. The paraformaldehyde may be added to the compound of formula II′ in 4 portions, 5 portions, 6 portions, 7 portions, or 8 portions, or more. For example, paraformaldehyde is added in four or more portions separated by at least about 10 minutes, by at least about 15 minutes, by at least about 20 minutes, by at least about 25 minutes, by at least about 30 minutes. In embodiments, the paraformaldehyde is added in six or more portions separated by at least about 30 minutes.

Alternatively, the paraformaldehyde is added to the compound of formula II′ over a period of at least about 45 minutes, or at least about 1 hour, at least about 1.5 hours, at least about 2 hours, at least about 2.5 hours, at least about 3 hours, at least about 3.5 hours, at least about 4 hours, at least about 4.5 hours, or at least about 5 hours, wherein the addition is done continuously over the time period (i.e., from the first addition to the last addition), for example by continuous or dropwise addition, or in multiple portions over the time period.

The acid used in the reaction mixture of Scheme 3 is a strong acid. In embodiments, the acid in the reaction of Scheme 3 may have a pKa of less than about 2.5, or a pKa of less than about 2.0, or a pKa of less than about 1.5, or a pKa of less than about 1, or a pKa of less than about 0.9, or a pKa of less than about 0.8, or a pKa of less than about 0.7, or a pKa of less than about 0.6, or a pKa of less than about 0.5. In embodiments, the acid in the reaction of Scheme 3 may have a pKa of less than about 0.9.

The acid in the reaction of Scheme 3 may include, but is not limited to, HF, HCl, HBr, HClO₄, HNO₃, H₂SO₄, H₃PO₄, a sulfonic acid such as methane sulfonic acid, trifluoromethane sulfonic acid, ethane sulfonic acid, benzene sulfonic acid or toluene sulfonic acid, Eaton's reagent, trifluoroacetic acid, or combinations thereof. In embodiments, the acid in the reaction of Scheme 3 includes HF, HCl or HBr, and particularly, the acid is HCl. The molar concentration of the acid used in this reaction may range from about 5 to about 10 M, from about 6 to about 9 M, or from about 7 to about 9 M.

Additional improvements during the synthesis of glycoluril dimer compound (IV) or (IV-A) with significantly improved purity and yield by optimizing the reaction temperature at about or higher than 55° C. Prior, during and after the addition of paraformaldehyde to the reaction according to Scheme 3, the temperature of the reaction vessel is elevated. The temperature of the reaction vessel after adding the paraformaldehyde is maintained at a temperature above 50° C., above 51° C., above 52° C., above 53° C., above 54° C., above 55° C., above 56° C., above 57° C., 58° C., above 59° C., above 60° C., above 61° C., above 62° C., above 63° C., above 64° C., above 65° C., above 66° C., above 67° C., above 68° C., above 69° C., or above 70° C.

Although heating the reaction of Scheme 3 could promote side reactions including oligomerization and polymerization that would reduce the yield of the glycoluril dimer of formula IV, the inventors have found that performing the reaction at an elevated temperature promoted an improved yield and purity of the reaction product.

The method may further include a step of isolation of the glycoluril dimer compound of formula IV. For example, the isolation may include recrystallisation, precipitation, aggregation, centrifugation, filtration or combinations thereof. The isolation process may include contacting the reaction mixture with water, heating to a temperature of at least about 70° C., cooling to a temperature below about 30° C., and isolating the solid, for example by filtering or centrifuging the reaction mixture.

After adding or contacting water to the reaction mixture, the reaction mixture is heated to the temperature of at least about 70° C., at least about 71° C., at least about 72° C., at least about 73° C., at least 74° C., at least about 75° C., at least about 76° C., at least about 77° C., at least about 78° C., at least about 79° C., or at least about 80° C. The reaction vessel can be set at the temperature at least about 70° C., at least about 71° C., at least about 72° C., at least about 73° C., at least 74° C., at least about 75° C., at least about 76° C., at least about 77° C., at least about 78° C., at least about 79° C., or at least about 80° C. while the reaction mixture is stirring for about 1 to 5 days, or for about 2 to 5 days, or for about 3 days.

After heating the reaction mixture and the water, the temperature of the reaction mixture is cooled below about 30° C., below about 29° C., below about 28° C., below about 27° C., below about 26° C., below about 25° C., below about 24° C., below about 23° C., below about 22° C., below about 21° C., or below about 20° C.

In certain embodiments, the compounds of formula IV in a solid form (e.g., precipitate, particles, or powders) may be collected by centrifuging the reaction mixture. Alternatively, the compounds of formula IV in a solid form (e.g., precipitate, particles, or powders) may be collected by filtering. In large scale synthesis, precipitation conditions after the reaction may be important to improve the production yield and purity of the reaction in Scheme 3. The precipitation may include adding various solvents (e.g., water or aqueous solution, or a mixture of polar solvent) and followed by removing the solid, for example by filtering.

A synthesis of glycoluril dimer 3 was disclosed by Isaacs and coworkers in “Acyclic cucurbit[n]uril molecular containers enhance the solubility and bioactivity of poorly soluble pharmaceuticals,” Nature Chem 4, 503-510 (2012). However, this procedure does not suggest a method for reducing the solidification of the reaction mixture, which the inventors have found to greatly complicate the purification and isolation of the dimethyl glycoluril 3. Additionally, the reported method is performed at a low temperature that the inventors have determined is disadvantageous for yield and purity of the glycoluril dimer. Furthermore, the inventors have found that the process of direct crystallization of the crude material as reported in previous literature provides reduced purity of the final product.

In embodiments, the methods include the following Scheme 4.

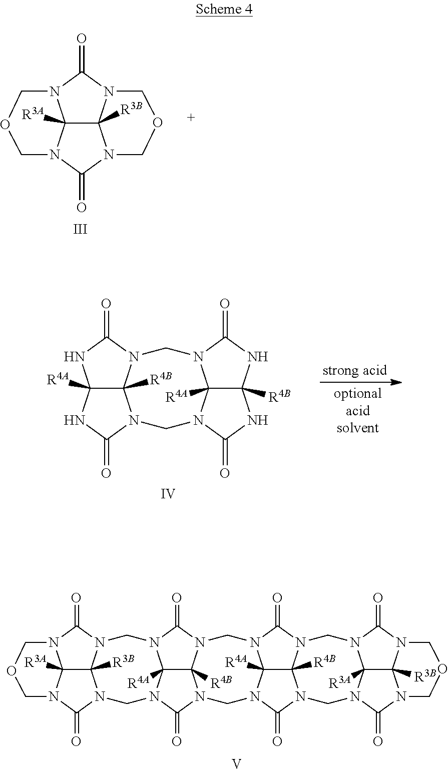

wherein R^{4A}, and R^{4B} are as described herein.

The reaction according to Scheme 4 involves the condensation of the glycoluril bis-ether of formula III and the glycoluril dimer of formula IV to obtain a glycoluril tetramer of Formula V. The reaction in Scheme 4 is performed in the presence of (a) one or more acids having a pKa of less than about 1, and optionally (b) one or more polar aprotic solvents and/or an additional acid solvents.

In particular, the reaction solvent includes TFA, which may be present as a sole solvent or may be combined with one or more polar aprotic solvents, in combination with one or more strong acids (e.g., Eaton's reagent). Using TFA without MeSO₃H or Eaton's reagent typically results in very slow and ineffective reaction. Most non-acidic solvents fail to fully dissolve the reagents and product or is incompatible with the strong acidic components.

In certain embodiments, a solid form of the glycoluril bis-ether of formula III may be dispersed in a solvent component and added to the reaction vessel containing the glycoluril dimer. Moreover, the reaction as described in Scheme 4 may require less amount of catalytic acid by using TFA. In certain embodiments, Eaton's reagent as one or more acids may still be added in about 10 equiv. or greater to promote an increase in reaction rate.

One or more of the acids used in the reaction mixture of Scheme 3 is a strong acid. In embodiments, the strong acid in the reaction of Scheme 4 may have a pKa of less than about 1, or less than about 0.5, or less than about 0, or less than about −1. The reaction mixture may comprise an additional acidic solvent. The additional acidic solvent in the reaction of Scheme 4 may have a pKa between about 3 to about 0, or may have a pKa of less than about 2.5, less than about 2.0, less than about 1.5, less than about 1, less than about 0.5.

The one or more strong acids in the reaction of Scheme 4 may include, but is not limited to, Eaton's reagent, MeSO₃H, toluene sulfonic acid, H₂SO₄, triflic acid, phosphoric acid, sulfuric acid, CSA, EtSO₃H, HCl, HBr, HClO₄, HNO₃, or combinations thereof. In embodiments, the acid includes methanesulfonic acid. In embodiments, the acid in the reaction includes Eaton's reagent, which is a combination of methanesulfonic acid and phosphorus pentoxide, for example including about 10 wt % phosphorus pentoxide in methanesulfonic acid. In embodiments, at least about 8 equivalents of the strong acid is used, and particularly, at least about 10 equivalents of the strong acid is used.

The solvents in the reaction of Scheme 4 may include an additional acid including, but is not limited to, trifluoroacetic acid (TFA), acetic acid, phosphoric acid, polyphosphoric acid (PPA), or combinations thereof. In certain embodiments, the additional acid is TFA.

In embodiments, a ratio between the one or more strong acids and the additional acid in the solvent may range from about 1:1 to 20:1, from about 1:1 to 19:1, from about 1:1 to 18:1, from about 1:1 to 17:1, from about 1:1 to 16:1, from about 1:1 to 15:1, or from about 1:1 to 14:1, from about 1:1 to 13:1, from about 1:1 to 12:1, from about 1:1 to 11:1, from about 1:1 to 10:1, from about 1:1 to 9:1, from about 1:1 to 8:1, from about 1:1 to 7:1, from about 1:1 to 6:1, from about 1:1 to 5:1, from about 1:1 to 4:1, from about 1:1 to 3:1, or from about 1:1 to 2:1.

Moreover, a ratio between the one or more strong acids and the additional acid in the solvent is about 20 to 1, about 19 to 1, about 18 to 1, about 17 to 1, about 16 to 1, about 15 to 1, about 14 to 1, about 13 to 1, about 12 to 1, about 11 to 1, about 10 to 1, about 9 to 1, about 8 to 1, about 7 to 1, about 6 to 1, about 5 to 1, about 4 to 1, about 3 to 1, about 2 to 1, or about 1 to 1.

The reaction of scheme 4 optionally includes one or more polar aprotic solvents in the reaction of Scheme 4 including, but not limited to, DMF, DMSO, acetonitrile, DMA, NMP, THF, dioxane, sulfolane, acetone, DMPU, diglymes, HMPA, or combinations thereof.

In embodiments, TFA is used as a solvent and about 10 equivalent of the strong acid (one or more acids having pKa less than 1, e.g., Eaton's reagent) in the reaction of Scheme 4.

During the reaction of Scheme 4, the temperature of the reaction vessel may be elevated due to less use of reactive solvent and acid mix by using TFA as solvents. The reaction temperature is maintained at a temperature above 55° C., above 56° C., above 57° C., above 58° C., above 59° C., above 60° C., above 61° C., above 62° C., above 63° C., above 64° C., above 65° C., above 66° C., above 67° C., above 68° C. In embodiments, the temperature of the reaction vessel is maintained at a temperature above 69° C., or above 70° C. In certain embodiments, the reaction temperature is maintained at a temperature at about 55° C.

Using these conditions, production yield may improve substantially, particularly, in a large scale reaction (e.g., kg scale reactions).

After the reaction is completed, remaining acids (e.g., MeSO₃H, etc.) may be removed by filtration and washing for quenching and before drying the crude solid. A single filtration/recrystallization step may not be sufficient for removing impurities including excess glycoluril bis-ether.

Further, as the reaction is completed, additional acid, such as TFA, may be added to the reaction, which can improve the precipitation and improve purification and/or yield in a large scale reaction. At the end of the reaction of Scheme 4, the compound of formula V may be isolated by the steps of adding additional acid, such as TFA, to the reaction mixture after completion of the reaction; precipitating the compound of formula V, for example by the addition of a countersolvent such as water; and collecting a mixture comprising the compound of formula V, for example by filtration.

The precipitation of the compound of formula V includes a step of adding a countersolvent for the compound of formula V. In certain embodiments, the countersolvent includes, but not limited to, water, methanol, ethanol, isopropyl alcohol, acetone, acetonitrile, THF, dioxane, and combinations thereof.

In embodiments, the compounds of formula V in a solid form (e.g., precipitate, particles, or powders) may be collected by centrifuging the mixture. Alternatively, the compounds of formula V in a solid form (e.g., precipitate, particles, or powders) may be collected by filtering. In embodiments, the compounds obtained may be washed with water and/or acetone.

A synthesis of glycoluril tetramer compound 5 is reported by Isaacs and coworkers (Nature Chem 4, 503-510 (2012)) where the investigators found a lower yield (36%). Additionally, the reported purification process of direct recrystallization of the crude product with TFA following a water wash was found to provide lower purity product. Other methods for formation of this compound have been published (see, e.g., Org. Chem. Front., 2019, 1555), however we have found that the procedures were not feasible at large scale due to the small particle size of the product penetrating through the filter centrifuge. Furthermore, we saw improved purity when reducing the concentration of Eaton's reagent along with waste disposal benefits and cost benefits associated with using less strong acid.

In one aspect, the methods of preparing the compounds of formula I include the following Scheme 1.

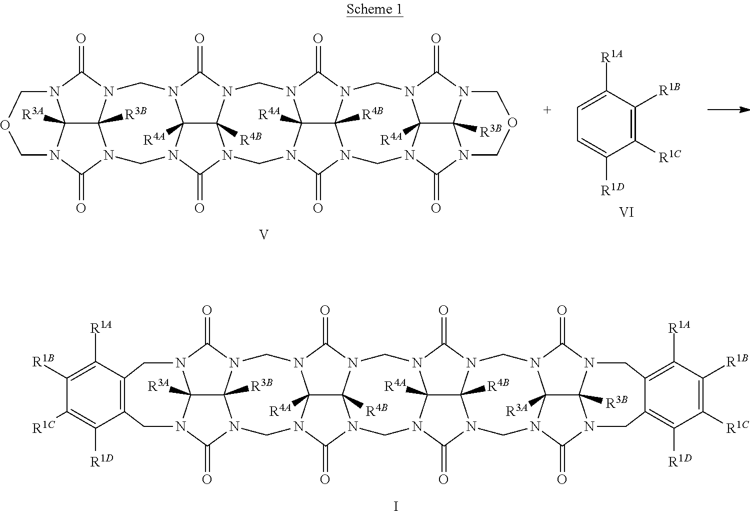

R^{1A}, R^{1B}, R^{1C}, R^{1D}, R^{3A}, R^{3B}, R^{4A}, and R^{4B} are as described herein.

The reaction according to Scheme 1 involves the condensation of the glycoluril tetramer of Formula V with the compound of Formula VI. The reaction in Scheme 1 is performed in the presence of a solvent system including (i) one or more acids having a pKa of less than about 1 and (ii) anhydride.

In embodiments, the one or more acids having a pKa of less than about 1 includes trifluoroacetic acid, Eaton's reagent, methane sulfonic acid (MeSO₃H), toluene sulfonic acid (TsOH), triflic acid, phosphoric acid, sulfuric acid, camphorsulfonic acid (CSA), ethane sulfonic acid (EtSO₃H), or combinations thereof. In embodiments, the one or more acids having a pKa of less than about 1 comprises trifluoroacetic acid.

Using the anhydride as a co-solvent greatly increases the reaction rate and overall yield of the reaction, e.g., overall yield (66%). The anhydride may be an organic acid anhydride in which the acyl groups of the acid anhydride are derived from carboxylic acids, sulfonic acids, phosphonic acids, or combinations. The anhydride may be selected from compounds having the general structure R′—C(═O)—O—C(═O)—R″ wherein R′ and R″ may me the same or different and are independently selected from alkyl, aryl, heteroalkyl, or may be taken together to form a heterocyclic ring, each of which may be substituted by halo, alkyl, trihaloalkyl, or the like. In certain embodiments, the anhydride may be selected from, but is not limited to, acetic anhydride, trifluoroacetic acid anhydride, butyric anhydride, propionic anhydride, triflic anhydride, succinic anhydride, maleic anhydride, or combinations thereof. The anhydride may be acetic anhydride, trifluoroacetic anhydride, or a combination thereof.

The amount of anhydride in the reaction may be from about 0.01% by volume of solvent (i.e., vol. anhydride/vol. total solvent×100) up to the concentration at which the anhydride is no longer soluble in the reaction solvent. The anhydride may be present from about 0.01% to about 60% by volume of solvent, or from about 0.3% to about 55% by volume of solvent, or from about 1% to about 55% by volume of solvent, or from about 2% to about 50% by volume of solvent; or from about 1% to about 45% by volume of solvent, or from about 2% to about 40% by volume of solvent.

The reaction in Scheme 1 can benefit by avoiding using an undue excess of compound VI such that overall synthesis is more efficient and economical. The reaction of Scheme 1 may use about 4 equivalents or less of compound of Formula VI, or about 3 equivalents or less of compound of Formula VI. In embodiments, the reaction of Scheme 1 uses from about 2 equivalents to about 5 equivalents of compound of Formula VI, or from about 2.5 equivalents to about 4.5 equivalents of compound of Formula VI, or from about 2.5 equivalents to about 4 equivalents of compound of Formula VI.

The resulting product after this reaction may be precipitated using antisolvents (e.g., a polar solvent), particularly in large scale synthesis. Exemplary antisolvents may include acetone, salt water, ethanol, methanol, brine, or combinations thereof.

The synthesis of cucurbituril compounds have been reported (see, e.g., Org. Biomol. Chem., 2014, 12, 2413-2422; WO2012/051407). However, the procedures report lower yields (e.g., 40% vs. 67%) and do not use an acid anhydride as a cosolvent, which we have found improves reaction speed in this synthesis. Furthermore, we have seen incomplete reactions using anhydride-free conditions, which leads to reduced purity and yield in the final product.

In one aspect, the methods of preparing the compounds of formula (I-A) include the following Scheme 5.

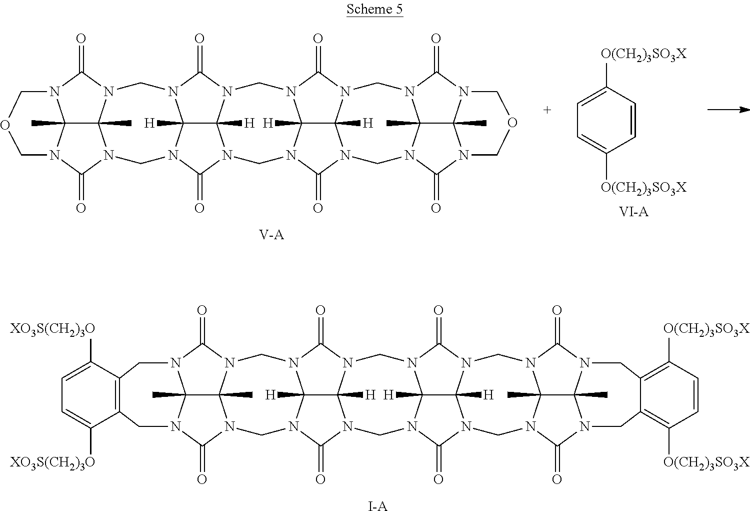

X is as described herein.

The reaction according to Scheme 5 includes contacting the compound of formula VI-A and a compound having the formula V-A, according to the conditions discussed above for Scheme 1.

In one aspect, the methods of preparing the compounds of formula (I-B) include the following Scheme 6.

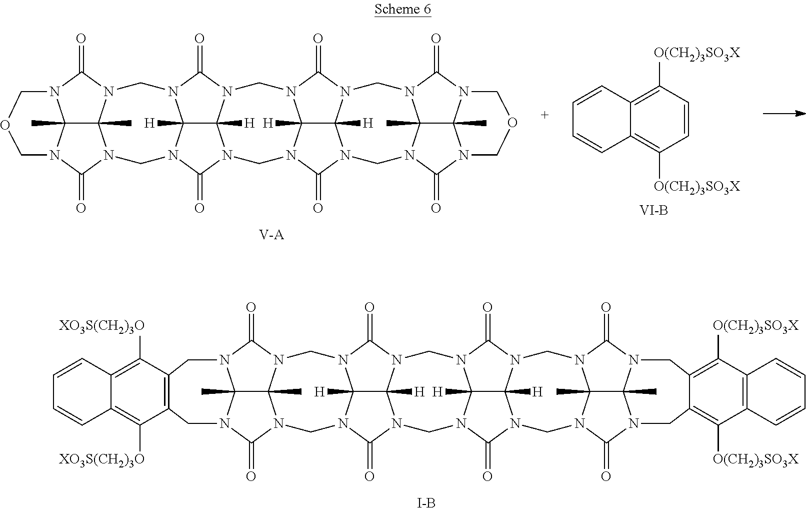

X is as described herein.

In embodiments, the methods include contacting the compound of formula VI-B and a compound having the formula V-A according to the conditions discussed above for Scheme 1.

In embodiments, the methods include the following Scheme 7 to produce compounds of formula VI-A.

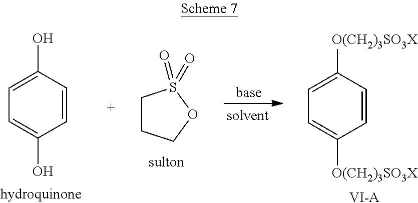

X is as described herein.

The reaction in Scheme 7 is performed in the presence of a base, and a solvent including one or more of water, polar aprotic solvents, and combinations thereof.

Instead of adding the base in an aqueous basic solution or as a solvent, the base may be added in a salt or solid form. As a consequence, reduced molar equivalents of the base may exist in reaction mixture, for example, the base is present at about 1.5 to 3.0 equivalents, at about 2.0 to 2.5 equivalents, at about 2.2 to 2.4 equivalents, at about 2.0 equivalents, at about 2.1 equivalents, or particularly at about 2.2 equivalents, at about 2.3 equivalents, at about 2.4 equivalents, or at about 2.5 equivalents. The base includes, but is not limited to, an alkali metal hydroxide (e.g., NaOH, LiOH, or KOH), or quaternary ammonium hydroxide.

The amount of base, such as NaOH, in this reaction can provide higher yields and purities for compound of formula (VI-A). While sultones are known to hydrolyze in the presence of base, the finding that the reaction rate of sultone hydrolysis competed with the alkylation of hydroquinone with sultone is not known. For example, conventionally, a large excess of NaOH (2.5M) may be used as a cosolvent in such reaction, however, excess of NaOH also reacts with 1,3-propane sultone leading to incomplete reaction and yield loss.

The sultone may be added over an extended period, e.g., added dropwise or by several portions. In embodiments, in the reaction of Scheme 6, the sultone is added over a period of greater than about 1 hour, about 1.5 hour, about 2 hour, about 2.5 hour, about 3 hour, about 3.5 hour, about 4 hour, about 4.5 hour, or about 5 hour. Alternatively, the sultone is added in two, three, four or more portions separated by at least about 30 minutes, or by at least about 1 hour. In embodiments, the sultone is added in four or more portions separated by at least about 1 hour, at least about 1.5 hour, at least about 2 hours, at least about 2.5 hours, or at least about 3 hours.

The reaction further comprises hydroquinone. Alternatively, the reaction may use 1,4-dihydroxynaphthalene.

The reaction vessel containing hydroquinone, the base, and solvent is cooled to less than about 10° C., less than about 9° C., less than about 8° C., less than about 7° C., less than about 6° C., less than about 5° C. prior to addition of the sultone, or prior to each addition of the sultone when the sultone is added in fractions or portions.

The base in the reaction is in an amount equivalent to or greater than an amount of the sultone. In certain embodiments, the base in the reaction is in an amount of 1 to 10 equivalent of the sultone, e.g., 2 equivalent, 3 equivalent, 4 equivalent, 5 equivalent, 6 equivalent, 7 equivalent, 8 equivalent, 9 equivalent, or 10 equivalent of the sultone. Moreover, the base may be added over an extended period, e.g., added dropwise or by several portions. In certain embodiments, the base is added over a period of greater than about 1 hour, about 1.5 hour, about 2 hour, about 2.5 hour, about 3 hour, about 3.5 hour, about 4 hour, about 4.5 hour, or about 5 hour. Alternatively, the base is added in two, three, four, or more portions separated by at least about 1 hour, at least about 1.5 hour, at least about 2 hours, at least about 2.5 hours, or at least about 3 hours.

A synthesis of aromatic sidewall compound VI-A or VIB has been reported (see, e.g., Ma, D. et al. Nature Chem 4, 503-510 (2012); WO2012/051407). However, the process disclosed herein provides improved yield over this step (90% compared to 81%). Furthermore, the process disclosed herein allows for a reduction in the reaction time (e.g., from 12 h to 2 h). Also, reduced levels of the monosubstituted impurity are observed when using the procedure disclosed herein.

In embodiments, the methods include the following Scheme 8 to produce compounds of formula VI-B. X is as described herein. In embodiments, the reaction in Scheme 8 may be performed according to conditions in Scheme 7 as described above.

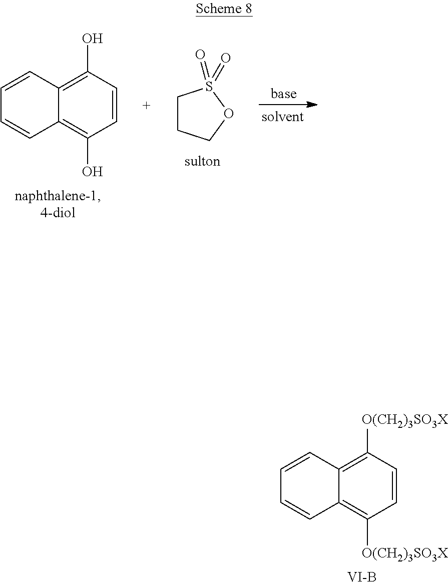

In embodiments, the methods include the following Scheme 9.

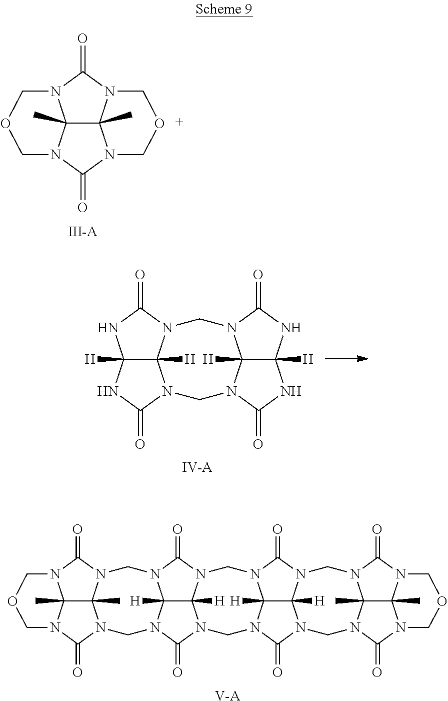

In embodiments, the reaction in Scheme 9 may be performed according to the conditions provided for Scheme 4 as described above.

In embodiments, the methods include the following Scheme 10.

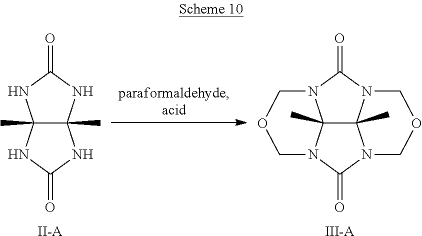

In embodiments, the reaction in Scheme 10 may be performed according to conditions for in Scheme 2 as described above.

In embodiments, the methods include the following Scheme 11.

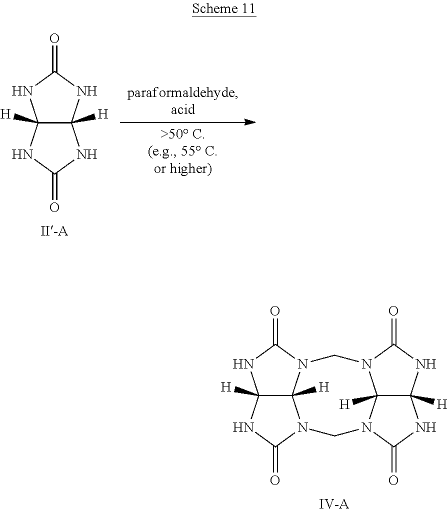

In embodiments, the reaction in Scheme 11 may be performed according to the conditions in Scheme 3 as described above.

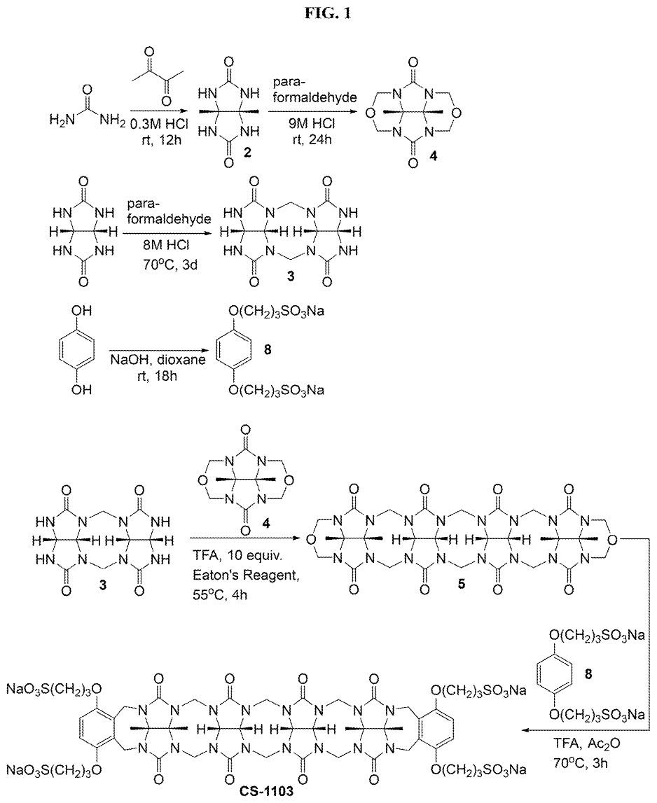

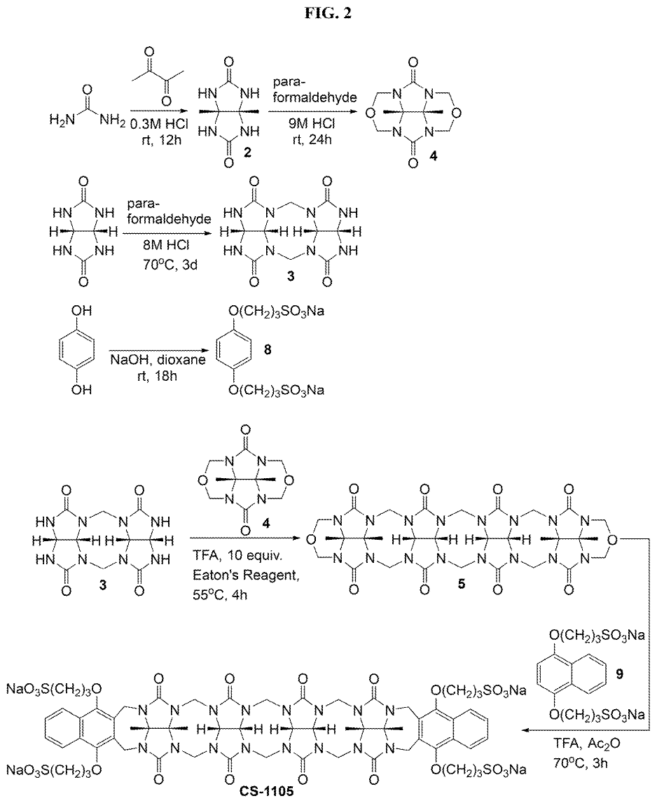

Exemplary synthesis of the compound of formula I-A (e.g., CS-1103) and formula I-B (e.g., CS-1105) are shown in FIG. 1 and FIG. 2, respectively.

Among other things, in Scheme 4, the reaction includes using trifluoroacetic acid (TFA) as the solvent and 10 equivalents of strong acids (e.g., Eaton's reagent, H₂SO₄, MsOH, TsOH, or camphor sulfonic acid) to promote reactivity. This significantly reduces the amount of excess sulfonic acid used compared to what has been reported. This process also has resulted in higher yields of compound of Formula V or V-A, and, unexpectedly, resulted in larger particles in the crude material which could be effectively filtered at scale. These advantages are unexpected benefits from switching solvent component in Scheme 4 to TFA in combination with strong acids (e.g., Eaton's reagent, H₂SO₄, methane sulfonic acid, toluene sulfonic acid, or camphor sulfonic acid, and the like). The use of TFA as a single solvent and catalyst without the addition of sulfonic acids was tried but found to be ineffective. In addition, the reduced viscosity of the reaction mixture of Scheme 4 was a surprising benefit of changing the reaction conditions. As consequence, significant optimization efforts may be eliminated while providing higher yield and purity. Further, the reaction mixture contains a lower concentration of the strong acid or Eaton's reagent, which allows for safer reaction conditions and easier disposal of waste. For example, workup process of the reaction is easier to perform with the weaker acid and the reaction mixture becomes less viscous which makes the reaction process easier to scale to large volumes.

According to certain embodiments, the reactions in Scheme 7 or 8, reduced the amount of NaOH in this reaction can provide higher yields and purities for compound of formula (VI-A) or (VI-B). While sultones are known to hydrolyze in the presence of base, the finding that the reaction rate of sultone hydrolysis competed with the alkylation of hydroquinone with sultone is not suggested in the related art. Conventionally, a large excess of NaOH (2.5M) is used in such reaction, however, excess of NaOH also reacts with 1,3 propane sultone leading to incomplete reaction and yield loss.

Moreover, according to certain embodiments, Scheme 3, the disclosure provides additional improvements during the synthesis of compound (IV) or (IV-A) with significantly improved purity and yield by optimizing the reaction temperature at about or higher than 55° C.

According to certain embodiments, in Scheme 1, the disclosure provides benefits in avoid using excess of compound VI or VI-A such that overall synthesis more efficient and economical.

### EXAMPLES

### Comparative Example A

Glycoluril Dimer [3]: Glycoluril (250 g, 1.76 mol) and paraformaldehyde (52.5 g, 1.76 mol) powders (no solvent) were stirred together in a Nalgene beaker with a metal spatula at room temperature until well mixed (˜5 minutes). An aqueous solution of HCl (8 M, 350 mL) was poured into the beaker slowly, while the mixture was stirred manually using a metal spatula. After −90 seconds of stirring a rubbery white solid was formed in the beaker. Heat was generated within the flask during this step (the beaker is warm to the touch, but the temperature was unmeasured). The white solid was immediately broken down into smaller pieces with a metal spatula and the pieces and a magnetic stir bar were added to a round bottom flask. The mixture was stirred at 50° C. for 1 h. Using a metal spatula, any large chunks of solid were broken up further. Residue along the sides of the flask were also scraped down into the mixture. This well-dispersed slurry was returned to the oil bath and stirred at 50° C. for 2 days.

The reaction mixture was then cooled to room temperature (RT) and filtered by vacuum filtration using Whatman 1 filter paper. Once mostly dry, the white semi-solid (glue-like) was transferred to an Erlenmeyer flask and stirred with 250 mL water. The mixture was stirred at RT overnight (until chunks were broken down into small particles). The slurry was filtered by vacuum filtration (Whatman 1 filter paper) and allowed time to dry on the filter paper (several hours) before transferring to the next step. If product is transferred before fully dry, then filter paper will stick to product during transfer and end up in a round-bottom (RB) flask. The crude solid (˜300 g) was transferred to a beaker, and 4×100 mL aliquots of TFA were added. After each aliquot, the TFA/crude slurry was added to a 1 L RB flask. The mixture was stirred at 75° C. for 2 h, then cooled to RT and left stirring at RT overnight. The mixture was filtered by vacuum filtration using Whatman 1 filter paper and dried on filter funnel for 1 day. The solid was dried until it was transferrable to a round bottom flask. To remove residual TFA, the dry solid was stirred at 80° C. with EtOH (600 mL) overnight.

The slurry was allowed to cool to RT and filtered by vacuum filtration using Whatman 1 filter paper and the solid was dried over the filter funnel for 1 day. The white solid was broken with a spatula and transferred to a RB flask then dried under high vacuum until a constant weight was reached (1 day). The resulting product was a white solid (119.7 g, 44%). Analysis was performed using ¹H and ¹³C NMR, melting point, and TR. The spectroscopic data matches that reported in the literature (Huang, W.-H.; Zavalij, P. Y.; Isaacs, L. J. Am. Chem. Soc. 2008, 130, 8446-8454).

### Comparative Example B

Glycoluril Tetramer Building Block [5]: Glycoluril dimer (13.1 g, 33.13 mmol) was dissolved in Eaton's Reagent (7.7% P₂O₅ in MeSO₃H, 95 mL) and the solution was heated to 50° C. under nitrogen. After 30 minutes, with good magnetic stirring the solid was mostly dissolved. Dimethyl glycoluril bis ether (4, 47.5 g, 186.4 mmol) was added in one portion, and a spatula was used to manually stir the powder into the solution.

The reaction mixture was stirred at 50° C. for 3 h. During the 3 hours, the reaction mixture increased in viscosity, so it is important to ensure that stirring is maintained. At 3 hours, the red solution was poured into 850 mL of water, and an additional 100 mL (950 mL total) was used to wash the interior of the flask into the total volume. The mixture was stirred at 25° C. for 10 minutes, then transferred to four 1 L centrifuge bottles and centrifuged (3000 RPM, 2500 G, 10 minutes), and the supernatant is decanted.

Water was added to each centrifuge bottle (150 mL each) and again the bottles were centrifuged and decanted (3000 RPM, 2500 G, 10 minutes) two additional times, until the decanted solvent was no longer pink. After decanting, the centrifuge tubes were held at −4° C. overnight for storage. The solid was dried by transferring into a round bottom flask and placed on high vacuum or rotovap. The dry solid was then dissolved in TFA (96 mL total, 24 mL in each centrifuge bottle). and bottles were vortexed or sonicated to facilitate dissolution. To the TFA solution, 150 mL water was added to each bottle resulting in the formation of a white precipitate. Samples were vortexed, then centrifuged (3000 rpm, 2500 G, 10 min) and decanted. The solid was then washed with water (600 mL to each bottle), vortexed or sonicated to break up the pellet, and centrifuged (4000 rpm, 4400 G, 10 min). The solid was then washed with acetone twice (2×) (300 mL to each tube), vortexed, and centrifuged (3800 rpm, 4000 G, 10 min). The precipitate was held at −4° C. overnight. The second round of TFA/water/acetone washes removes a colored impurity which cannot be seen on NMR. It is not clear how much impurity is removed compared to product.

The precipitate from each bottle was collected in a round bottom flask and centrifuge tubes were rinsed with EtOH in order to transfer all product to the flask. Rotary vaporization was used to remove the EtOH and the final product was dried under high vacuum. The combined sample was purified again dissolving all the solid in TFA (48 mL). The dissolved product in TFA was then added to six 50 mL falcon tubes in equal portions (8 mL in each centrifuge tube). The falcon tubes were filled with water to the 50 mL total, and centrifuged (3800 RPM, 2300 G, 10 minutes) and decanted. The falcon tubes were then filled with water to the 25 mL marking, centrifuged (3800 RPM, 2300 G, 10 minutes) and decanted. The solid was then washed by twice adding acetone to the centrifuge tubes up to the 25 mL marking, centrifuging the tubes (3800 RPM, 2300 G, 10 minutes) and decanting. The solid was transferred to a round bottom flask and dried using high vacuum until a constant weight was reached. The final product was a white powder (12.1 g, 47% yield).

Analysis was performed using ¹H and ¹³C NMR, melting point, and IR.

### Comparative Example C

Aromatic Sidewall Building Block [8]: A solution of hydroquinone (72.7 g, 0.66 mol) in aqueous NaOH solution (2.5 M, 1.0 L) was treated with a solution of 1,3-propanesultone (200 g, 1.64 mol) in 1,4-dioxane (1.0 L). The mixture was stirred at RT for 12 h.

This solution was then poured into acetone (4.0 L) and a reddish solid precipitated. This solid was collected via vacuum filtration (Whatman 1 filter paper) funnel and the solid was rinsed with 100 mL additional acetone while still in the Buchner funnel. The solid is transferred to a round bottom flask where it was dried overnight under vacuum. The solid was recrystallized with a mixed solvent of water and EtOH to yield the product as beige crystals (195 g, 74%). Note: 100 g of crude material was dissolved in 500 mL water, then 1.0 L EtOH was added. The mixture was heated to dissolve all the solid, and if necessary additional H₂O was added to facilitate this process. The mixture was then cooled down to RT. The recrystallized product was then collected by filtration.

Analysis was performed using ¹H and ¹³C NMR, melting point, and IR.

### Comparative Example D

CS-1103 synthesis: A solution of methyl tetramer (5) (20.0 g, 25.6 mmol) was formed in TFA/Ac₂O (v/v=1:1, 200 mL). To this, [8](C₆H₄(OCH₂CH₂CH₂SO₃Na)₂, 39.6 g, 102.4 mmol) was mixed in under stirring. The mixture was stirred and heated at 70° C. for 3 h.

The reaction mixture was poured into an Erlenmeyer flask with stirring MeOH (1.0 L) while still hot. The solid was collected by filtration (Whatman 1 filter paper) and was dried under high vacuum. Once dry, the solid (59.8 g) was dissolved in water (150 mL), stirred at room temperature, and precipitated using acetone (300 mL), and filtered using vacuum filtration. Once dry, the material was again dissolved in water (150 mL), and precipitated using acetone (300 mL), which resulted in the collection of 48.4 g of crude material. This material was dissolved in water (60 mL), stirred at room temperature, and precipitated using acetone (60 mL). The mixture was then cooled to 0° C. overnight and filtered using vacuum filtration (Whatman 1 filter paper). The resulting solid (˜23.5 g) was dissolved in water (94 mL) to a concentration of 250 mg/mL. This solution was filtered using Whatman 1 filter paper and the filtrate was adjusted to pH=7 by adding 1 M aqueous NaOH (˜150 μL). The filtrate was concentrated by rotary evaporation and then the solid was further dried under high vacuum to yield CS-1103 (23.4 g, 60%) as a pale yellow powder. This powder was 93-95% pure by HPLC-UV/Vis analysis.

### Recrystallization

The pale yellow powder CS-1103 (1.0 g) was suspended in deionized water (1.4 mL). The mixture was heated to reflux with stirring. When the compound was completely dissolved (˜10 minutes) the mixture was removed from heating and allowed to cool to room temperature slowly over two hours. Crystals began forming as the solution cooled. The solution containing the crystals was further cooled to 4° C. overnight. The resulting crystals were filtered and washed with ice cold deionized water. Approximately 400 mg (40%) of product was recovered as colorless crystals (>98% purity by HPLC UV-Vis analysis).

### Example 1: Synthesis of CS-1103

### Step 1: Synthesis of [2], Dimethyl glycoluril

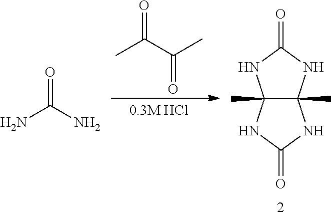

A solution of urea (1510 g, 25.1 mol) in HCl (0.3 M, 3.7 L) was treated with 2,3-butanedione (660 g, 7.7 mol). The solution was stirred at RT for 12 h. The reaction mixture was filtered and the solid was washed with water (3.5 L×2) and then ethanol (2.0 L) to yield 2 as a white solid (1168 g, 86%).

### Step 2: Synthesis of [4], dimethyl glycoluril bis-ether

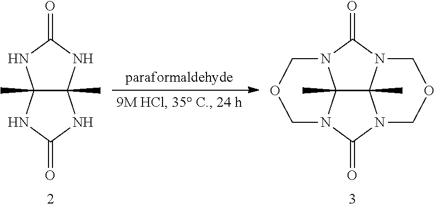

7.5 kg water and 66.86 kg 31.5% HCl were charged to the reactor and the reactor temperature was set to 35° C. A wet cake of dimethyl glycoluril (19.54 kg, 114.8 mol) was added to the reactor and paraformaldehyde (15.67 kg, 522.3 mol, 4.5 equiv.) was subsequently added and rinsed with water. The reaction mixture was set to a temperature of 50° C. and then cooled to a temperature of 35° C. The reaction mixture was stirred for 24 hours and then cooled to a temperature of 25° C. Water (120.5 L) was added for quenching and the resulting mixture was stirred at a temperature of 25° C. for 6 h, and filtered. The filtered mixture was washed with water (4×46.0 L), filtered and washed with acetone (0-2×27.6 L). The mixture was dried and 19.5 kg of glycoluril bis-ether was recovered at yield 72%.

### Step 3: Synthesis of [8], Aromatic Sidewall

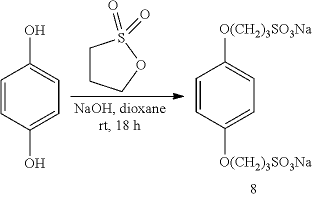

Hydroquinone (7.0 kg, 63.6 mol), water 48.7 kg, 20% NaOH (28.98 kg, 144.9 mol NaOH, 2.3 equiv.) and 12.34 kg water were charged to a reaction vessel. The reaction mixture is cooled to a temperature of 5° C. 20% of 1,3-propanesultone in dioxane (77.73 kg, 127 mol and 2.0 equiv. of sultone were added and then the reaction temperature was set to a temperature of 25° C., and the reaction mixture was stirred for 2 hours. After the reaction, the mixture was cooled to a temperature of 5° C.

20% 1,3-propanesultone in dioxane (19.41 kg, 31.8 mol, 0.5 equiv.) was added to the reaction product and heated to a temperature of 25° C. and stirred for 2 hours. Optionally, 20% 1,3-propanesultone in dioxane (19.41 kg, 31.8 mol, 0.5 equiv.) was added and stirred for 2 h. The reaction product was cooled to a temperature of 5° C., and 20% NaOH in water (13.04 kg, 65.2 mmol, 1.0 equiv.) was added, and these reaction was heated to a temperature of 25° C., and stirred for 2.5 hours. The reaction mixture was centrifuged, washed with acetone (2×53 L), and dissolved in water (35 kg). Additional EtOH (70 L) was added and heat-refluxed for 2 hours. The mixture was cooled to a temperature of 25° C., and then centrifuged, washed with acetone (2×53 L). The recrystallization was optionally repeated. The washed mixture was dried under vacuum at a temperature of 50° C. and 22.7 kg of compound 8 was recovered (90%).

### Step 4: Synthesis of [3], Glycoluril Dimer

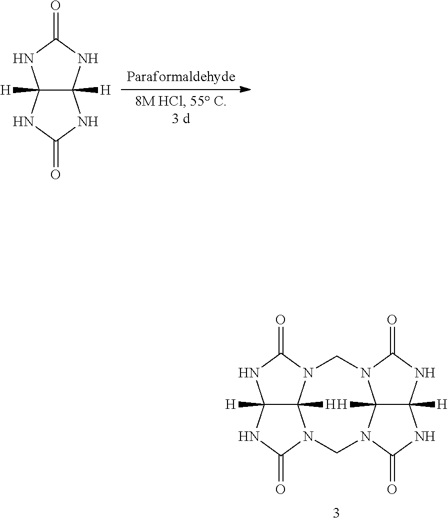

Water (8.8 kg), 31.5% HCl (35.8 kg), and 29.29 kg glycoluril (206.1 mol) were charged to a reaction vessel, and the reaction vessel was set to a temperature of 50° C. Paraformaldehyde (6.08 kg, 202.7 mol, 1.0 equiv.) was added into the reaction vessel in 6 parts and the reaction temperature was set to 70° C. and the reaction mixture was stirred for 3 days. The reaction mixture was diluted with 29.4 kg water and transferred to another vessel containing 119.4 kg water, and the vessel was set to a temperature of 80° C. and the reaction mixture was stirred for 2 hours. The reaction was cooled to a temperature of 25° C. and the reaction mixture is centrifuged, washed with water (2×115 L) and acetone 1×(73 L), dried under vacuum at a temperature of 50° C. 16.5 kg of the glycoluril dimer 3 was recovered (52%).

### Step 5: Synthesis of [5], Glycoluril tetramer

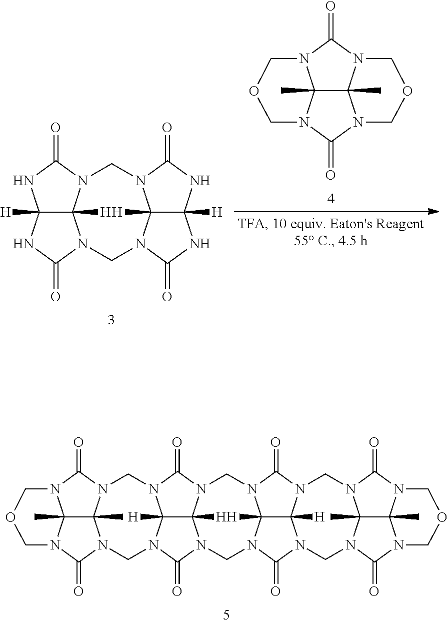

5.21 (20.5 mol) kg of dimer 3, 15.04 kg bis-ether 4 (59.2 mol, 2.8 equiv.), 41.78 kg TFA were charged into the reaction vessel and the reaction temperature was set to a temperature of 55° C., and stirred for 90 minutes until all solids were dissolved and the temperature was set to a temperature of 40° C. Eaton's reagent (19.31 kg) was added to the reaction vessel and the temperature was set to 55° C. for 4 hours, and then to 40° C. The reaction mixture was transferred to a vessel containing 148.4 kg water, set at a temperature of 25° C., and stirred for 30 minutes. The reaction mixture was centrifuged, washed with water (2×26.1 L) then acetone (2×25.5 L), transferred to vacuum oven, and then dried at a temperature of 50° C.

The dried material was dissolved in TFA (117.6 kg), and stirred at a temperature of 35° C. until dissolved, then water 83.66 kg was added. The mixture was centrifuged, washed with water (1×87.5 L) then acetone (1×88.2 L). Water (43 kg) was added to the mixture to create a slurry and it was centrifuged, washed with water (116 kg), then acetone (117 L). A slurry was created by adding 88 L acetone and the slurry was stirred, centrifuged, washed with acetone (117 L), and dried under vacuum at a temperature of 50° C. The reaction product 5 (15.0 kg) was recovered (51%).

### Step 6: Synthesis of CS-1103

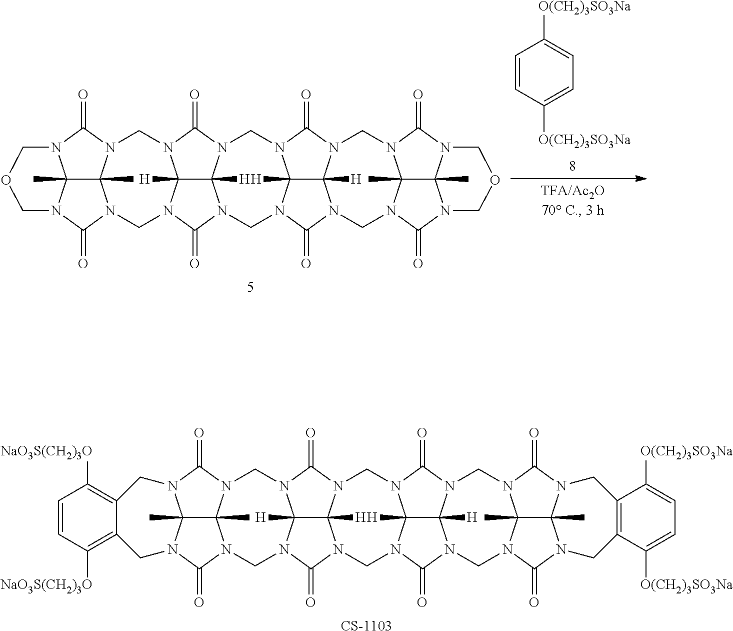

23.85 kg acetic anhydride, 26.81 kg TFA, compound 8 (10.23 kg, 25.7 mol, 4 equiv.), and tetramer 5 (4.99 kg) were charged into a reaction vessel. The reaction mixture was stirred at a temperature of 70° C. for 3 hours and cooled to a temperature of 35° C. Then, the reaction mixture was added to another vessel containing 80 kg MeOH, and the mixture was stirred for 30 minutes at a temperature of 0° C., centrifuged, and washed with MeOH (31.2 kg×2). The resulting product was dissolved in 37.5 kg water at a temperature of 50° C., then 60 kg acetone was added to precipitate the product. The product was centrifuged, washed with acetone (1×30.65 kg), dissolved in 38.5 kg water at a temperature of 50° C. Acetone (59.24 kg) was added to precipitate the product, and the mixture was centrifuged, washed with acetone (1×30.39 kg), dissolved in 27.5 kg water at a temperature of 50° C. 30 kg EtOH was added to the product mixture, stirred at a temperature of 15° C. for 1 hour, centrifuged, washed with EtOH (1×29.56 kg), dissolved in 30.22 kg water at a temperature of 50° C. 2% NaOH was further added until pH reached about 10.0, and the reaction mixture was filtered, filtered, precipitated with 47.27 kg EtOH, centrifuged, and dried in vacuum oven at a temperature of 50° C. CS-1103 of 11.8 kg (67%) was recovered.

### Example 2: Synthesis of CS-1105

The Synthesis scheme of CS-1105 is demonstrated in FIG. 2.

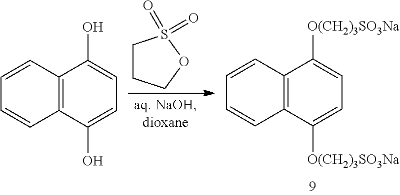

1,4-Hydroxynapthalene (30 g, 187.3 mmol) was dissolved in a solution of aqueous NaOH (18.6 g, 0.47 mol, in 250 mL of H₂O). In a separate flask, 1,3-propane sultone (57.2 g, 0.47 mol) was dissolved in dioxane. The solution containing 1,3-propane sultone in dioxane was added to the solution of aqueous 1,4 dihydroxy naphthalene. The reaction mixture was stirred at rt for 18 h. An additional 25 g (0.20 mol) of 1,3-propane sultone in dioxane (75 mL) was added to the reaction mixture. The reaction mixture was stirred at room temperature for 2 h. A solution containing 10 g NaOH (0.25 mol) in 50 mL H₂O was added to the reaction mixture to hydrolyze excess sultone. The reaction mixture was stirred for 2 h at rt and poured into 1 L of acetone. The resulting precipitate was filtered and dried on hi-vac. The precipitate was redissolved in H₂O (150 mL) and precipitated with 1.2 L of acetone. The resulting precipitate was filtered and dried on hi-vac to yield compound 9 as an off-white solid (77.0 g, 92%).

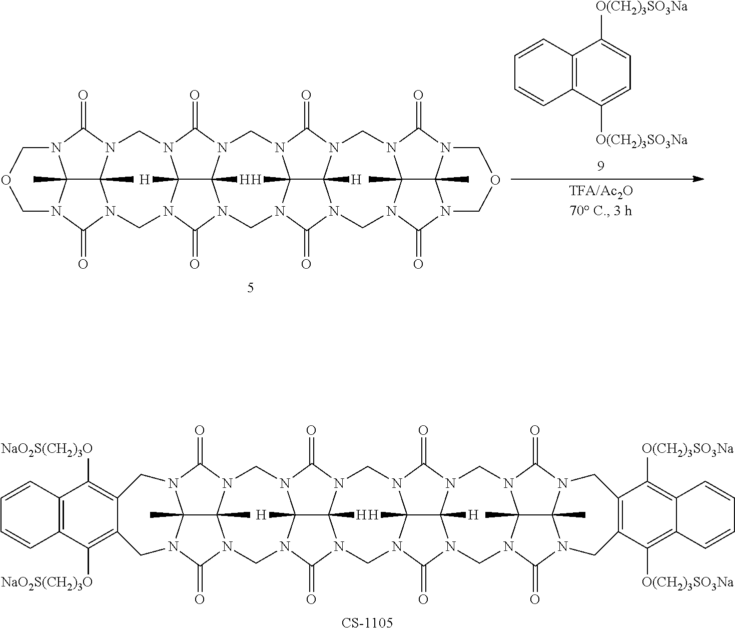

To a solution of glycoluril tetramer (30.00 g, 38.4 mmol) in TFA/Ac₂O (1.1, 250 mL) was added to aromatic sidewall 9 (68.2 g, 154 mmol). The mixture was stirred and heated at 70° C. for 3 h and poured into MeOH (1.5 L). The resulting suspension was stirred for 10 minutes and the precipitated solid was collected via filtration. The solid was recrystallized with the mixture of water and acetone (1:2, v/v, 750 mL, the crude material was first dissolved in water, and then acetone was added and the product was precipitated down from the solution) and then the crude product was recrystallized with a mix solvent of water (200 mL) and EtOH (200 mL) (Solid was dissolved in water with heating first, and then EtOH was added, and the mixture was cooled to 4° C.). The solid was dissolved in water (1.05 L) and filtered to remove insoluble impurities. The filtrate was adjusted to pH=7 by adding 1 M aqueous NaOH. The solvent was removed and then the solid was further dried under high vacuum to yield CS-1105 as an off-white solid (43.8 g, 69%).

### Example 3

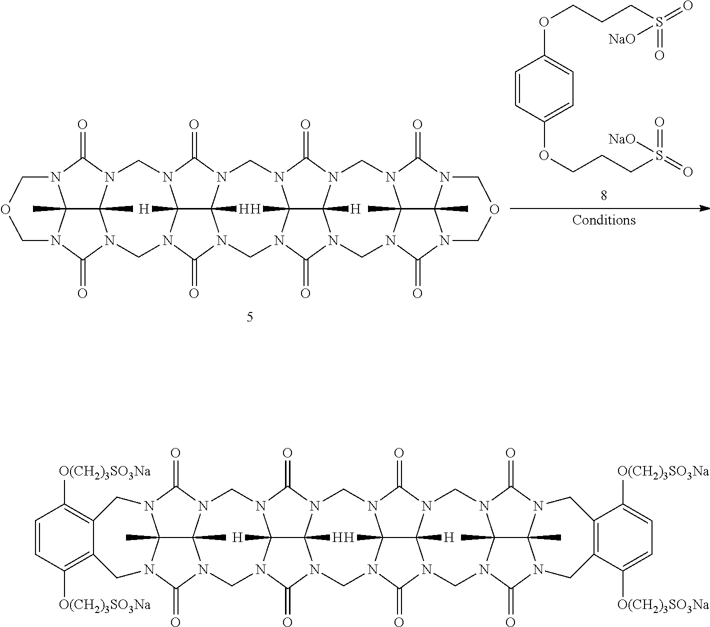

The effect of varying the amount of acetic anhydride in the above reaction was studied. Table 1 provides the conditions used for this study.

TABLE 1

| | Comp 5 | Comp 5 | Comp 8 | | Ac₂O | MeSO₃H | Temp | |
|---|---|---|---|---|---|---|---|---|
| Rxn # | (mg) | (mmol) | (mg) | TFA (μL) | (μL) | (μL) | (° C.) | purity |
| A | 50 | 0.0640 | 102 | 300 | 300 | 0 | 70 | 80.6 |
| B | 50 | 0.0640 | 102 | 375 | 225 | 0 | 70 | 86.6 |
| C | 50 | 0.0640 | 102 | 450 | 150 | 0 | 70 | 84.7 |
| D | 50 | 0.0640 | 102 | 525 | 75 | 0 | 70 | 87.0 |
| E | 50 | 0.0640 | 102 | 585 | 15 | 0 | 70 | 85.8 |
| F | 50 | 0.0640 | 102 | 600 | 0 | 0 | 70 | 82.8 |
| G | 50 | 0.0640 | 102 | 0 | 0 | 600 | 70 | — |
| H | 50 | 0.0640 | 102 | 0 | 0 | 600 | rt | 74.5 |

Solvent (i.e., TFA, acetic anhydride, MeSO₃H), compound 8, and tetramer compound 5 were charged into an 8 mL reaction vessel. The reaction mixture was stirred at a temperature of 70° C. for 4 hours. A 10 μL sample of each reaction was removed and added to 1 mL of DMSO and the relative product purity was determined by HPLC. Each of the reactions A through E show good relative purity, with reactions B to E showing improved relative purity.

### Example 4

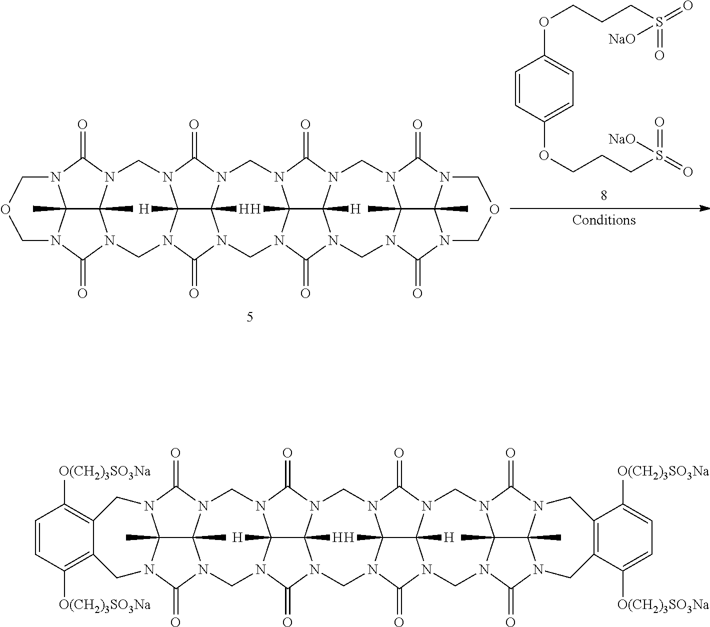

The effect of varying the amount of trifluoroacetic anhydride (TFAA) in the above reaction was studied. Table 2 provides the conditions used for this study.

TABLE 2

| | Comp 5 | Comp 5 | Comp 8 | TFA | Ac₂O | TFAA | Temp | |
|---|---|---|---|---|---|---|---|---|
| Rxn # | (mg) | (mmol) | (mg) | (μL) | (μL) | (μL) | (° C.) | Comment |
| A | 50 | 0.0640 | 77 | 1000 | 30 | 0 | 70 | |
| B | 50 | 0.0640 | 77 | 1000 | 0 | 30 | 70 | |
| C | 50 | 0.0640 | 77 | 500 | 0 | 500 | 70 | |
| D | 50 | 0.0640 | 77 | 1000 | 30 | 30 | 70 | |
| E | 50 | 0.0640 | 77 | 1000 | 30 | 30 | 70 | Stir for 30 min then add |
| | | | | | | | | comp. 8 |
| F | 50 | 0.0640 | 77 | 1000 | 30 | 30 | 70 | Stir for 90 min then add |
| | | | | | | | | comp. 8 |
| G | 50 | 0.0640 | 77 | 1000 | 0 | 30 | 70 | Stir for 30 min then add |
| | | | | | | | | comp. 8 |
| H | 50 | 0.0640 | 77 | 500 | 0 | 30 | rt | Stir for 90 min then add |
| | | | | | | | | comp. 8 |

Solvent (i.e., TFA, acetic anhydride, TFAA), compound 8, and tetramer compound 5 were charged into an 8 mL reaction vessel according to the parameters in Table 2. The reaction mixture was stirred at a temperature of 70° C. for a total of 4 hours. A 10 μL sample of each reaction was removed and added to 1 mL of DMSO and the relative product purity was determined by HPLC. Each of the reactions A through E show good relative purity, with reactions C and D showing improved relative purity. Reactions E to H showed reduced relative purity.

### Example 5

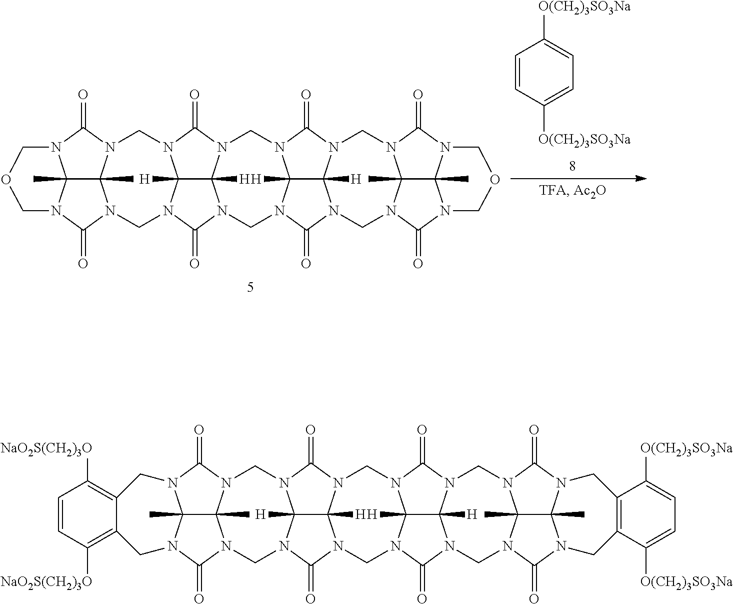

The effect of varying the reaction temperature in the above reaction was studied. Table 3 provides the conditions used for this study.

TABLE 3

| | Comp 5 | Comp 5 | Comp 8 | TFA | Ac₂O | Temp |
|---|---|---|---|---|---|---|
| Rxn # | (mg) | (mmol) | (mg) | (μL) | (μL) | (° C.) |
| A | 50 | 0.0640 | 77 | 1000 | 30 | 60 |
| B | 50 | 0.0640 | 77 | 1000 | 30 | 50 |
| C | 50 | 0.0640 | 77 | 1000 | 30 | rt (~20) |

Solvent (i.e., TFA, acetic anhydride), compound 8, and tetramer compound 5 were charged into an 8 mL reaction vessel. The reaction mixture was stirred at the temperature indicated in Table 3 for 4 hours. A 10 μL sample of each reaction was removed and added to 990 μL of DMSO and the relative product purity was determined by HPLC. Each of the reactions showed decrease relative product purity as compared to the reaction run at 70° C.

### Example 6

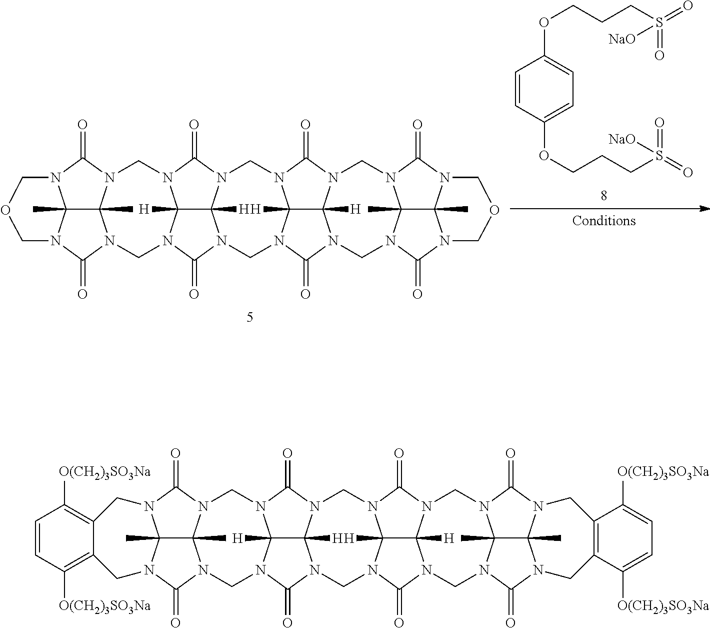

| Rxn | Comp 5 | Comp 5 | Comp 8 | TFA | Ac₂O | Temp | | |
|---|---|---|---|---|---|---|---|---|
| # | (mg) | (mmol) | (mg) | (μL) | (μL) | (° C.) | purity | Comment |
| A | 50 | 0.0640 | 102 | 193 | 220 | 70 | 80.3 | |
| B | 50 | 0.0640 | 102 | 1000 | 1000 | 70 | 78.2 | conc. was reduced and precip |
| | | | | | | | | in MeOH, dissolve precip and |
| | | | | | | | | HPLC |
| C | 50 | 0.0640 | 77 | 1000 | 30 | 70 | 86.7 | 3 eq. Comp 8; 5 eq Ac₂O |
| D | 50 | 0.0640 | 102 | 2000 | 30 | 70 | 86.9 | 4 eq. Comp 8; reduced conc.; |
| | | | | | | | | reduced Ac₂O |
| E | 50 | 0.0640 | 204 | 2000 | 61 | 70 | 85.0 | 8 eq. Comp 8; low con.; |
| | | | | | | | | reduced Ac₂O |

Solvent (i.e., TFA, acetic anhydride), compound 8, and tetramer compound 5 were charged into a 4 mL reaction vessel. The reaction mixture was stirred at 70° C. for 4 hours. A 10 μL sample of each reaction was removed and added to 990 μL of DMSO and the relative product purity was determined by HPLC. Each of the reactions A through E show good relative purity, with reactions c to E showing improved relative purity.

What is claimed:

1. A method for the preparation of a compound having the formula I 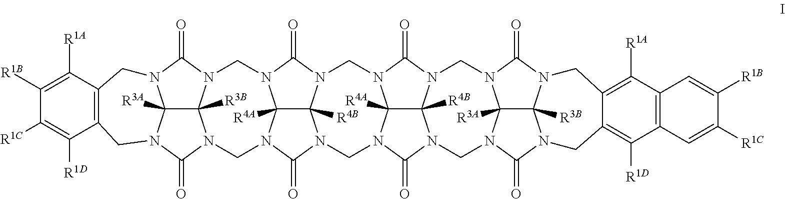
or a pharmaceutically acceptable salt thereof,
wherein:
each R^{1A} and R^{1D} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹;
each R^{1B} and R^{1C} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹; or,
additionally or alternatively, two R^{1A}, R^{1B}, R^{1C} and R^{1D} attached on the same phenyl ring at adjacent positions, together with atoms to which they are attached, are joined to form a fused C₆-C₁₂ aryl, 5 to 12 membered heteroaryl, or 5 to 7 membered heterocycle, which are optionally substituted with 1 to 3 substituents independently selected halogen, —OH, —NH₂, substituted or unsubstituted C₁-C₆ alkyl, or substituted or unsubstituted 2 to 6 membered heteroalkyl
each R^{3A} and R^{3B} is independently selected from hydrogen, halogen, —OH, C₁-C₆ alkyl, phenyl, substituted phenyl and 2 to 6 membered heteroalkyl;
each R^{4A} and R^{4B} is independently selected from hydrogen, halogen, —OH, C₁-C₆ alkyl, phenyl, substituted phenyl and 2 to 6 membered heteroalkyl;
each n1 is independently selected from 0 to 5;
each v1 is independently selected from 2 or 3; and
each X¹ is independently selected from selected from H, —OH, C₁-C₆ alkyl, alkali metal cation, and quaternary ammonium cation;
the method comprising:
(i) contacting a compound having the formula II 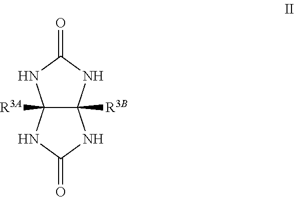
and paraformaldehyde in the presence of acid to provide a compound having the formula III 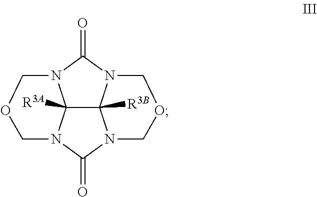
(ii) contacting a compound having the formula II′ 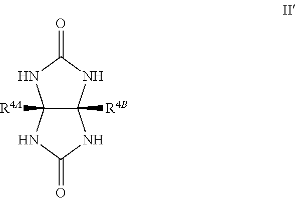
with paraformaldehyde and acid, wherein the paraformaldehyde is added to the compound of formula II′ over a period of at least about 1 hour, and wherein the reaction is maintained at a temperature above 55° C., to provide a compound having the formula IV 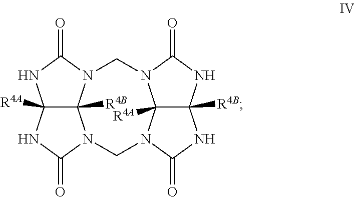
(iii) contacting a compound having the formula III 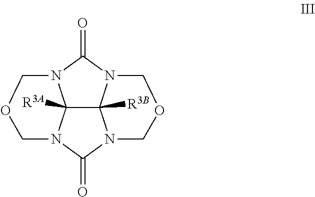
and a compound having the formula IV in the presence (a) of one or more acids having a pKa of less than about 1, and optionally (b) one or more polar aprotic solvents and/or additional acid solvents,
to provide a compound having the formula V: 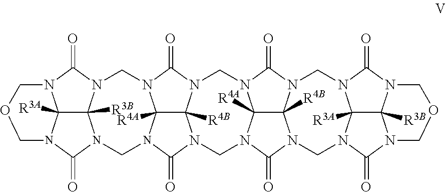
and isolating the compound of formula V;
(iv) contacting the compound having the formula V and a compound having the formula VI 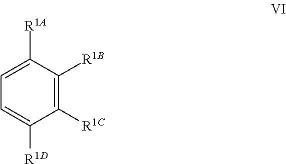
in the presence of a solvent comprising one or more acids having a pKa of less than about 1 and anhydride to provide the compound having the formula I.

2. The method of claim 1, wherein the reaction of step (i) is maintained at a temperature above about 35° C.

3. The method of claim 1 or claim 2, wherein in the reaction of step (ii) the paraformaldehyde is added in four or more portions separated by at least about 10 minutes, or by at least about 15 minutes, or by at least about 20 minutes, or by at least about 25 minutes, or by at least about 30 minutes.

4. The method of claim 3, wherein in the reaction of step (ii) the paraformaldehyde is added to the compound of formula II′ in six or more portions separated by at least about 30 minutes.

5. The method of claim 1 or claim 2, wherein in the reaction of step (ii) the paraformaldehyde is added by continuous or dropwise addition over a period of at least about 45 minutes, or at least about 1 hour, at least about 1.5 hours, at least about 2 hours, at least about 2.5 hours, at least about 3 hours, at least about 3.5 hours, at least about 4 hours, at least about 4.5 hours, or at least about 5 hours.

6. The method according to any one of claims 1 to 5, wherein the reaction of step (ii) is maintained at a temperature above 65° C.

7. The method according to any one of claims 1 to 6, wherein in the reaction of step (ii) the acid has a pKa of less than about 2.5, or a pKa of less than about 2.0, or a pKa of less than about 1.5, or a pKa of less than about 1, or a pKa of less than about 0.9, or a pKa of less than about 0.8, or a pKa of less than about 0.7, or a pKa of less than about 0.6, or a pKa of less than about 0.5. In embodiments, the acid in the reaction of Scheme 3 may have a pKa of less than about 0.9.

8. The method according to any one of claims 1 to 7, wherein in the reaction of step (ii) the acid is selected from the group consisting of HF, HCl, HBr, HClO₄, HNO₃, H₂SO₄, H₃PO₄, a sulfonic acid such as methane sulfonic acid, trifluoromethane sulfonic acid, ethane sulfonic acid, benzene sulfonic acid or toluene sulfonic acid, Eaton's reagent, trifluoroacetic acid, or combinations thereof.

9. The method according to any one of claims 1 to 7, wherein in the reaction of step (ii) the acid is HCl.

10. The method according to any one of claims 1 to 9, wherein the reaction mixture of step (ii) does not solidify during the reaction process.

11. The method according to any one of claims 1 to 10, wherein the method further comprises a step of isolating the compound of formula IV in step (ii), which comprises contacting the reaction mixture with water, heating to a temperature of at least about 70° C., cooling to a temperature below about 30° C., and centrifuging or filtering the reaction mixture.

12. The method according to any one of claims 1 to 11, wherein the reaction temperature of step (iii) is maintained at or above about 55° C.

13. The method according to any one of claims 1 to 12, wherein the solvent of step (iii) comprises an acid selected from the group consisting of trifluoroacetic acid, phosphoric acid, sulfuric acid, HCl, HBr, HClO₄, HNO₃, triflic acid, methane sulfonic acid, toluene sulfonic acid, Eaton's reagent, and combinations thereof.

14. The method according to any one of claims 1 to 13, wherein the polar aprotic solvent of step (iii) comprises DMF, DMSO, acetonitrile, DMA, NMP, THF, dioxane, sulfolane, acetone, DMPU, diglymes, HMPA, or combinations thereof.

15. The method according to any one of claims 1 to 14, wherein step (iii) comprises a solvent which includes trifluoroacetic acid.

16. The method according to any one of claims 1 to 15, wherein the method further comprises a step of isolating the compound of formula V, the isolating step comprises:
adding additional TFA to the reaction mixture after completion of the reaction;
precipitating the compound of formula V; and
filtering a mixture comprising the compound of formula V.

17. The method of claim 16, wherein the step of precipitating the compound of formula V comprises adding a countersolvent for the compound of formula V, and the countersolvent comprises methanol, ethanol, isopropyl alcohol, acetone, acetonitrile, THF, dioxane, or combinations thereof.

18. The method of any one of claims 1 to 17, wherein in step (iii) or (iv), the one or more acids having a pKa of less than about 1 comprises methane sulfonic acid, toluene sulfonic acid, triflic acid, trifluoroacetic acid, phosphoric acid, sulfuric acid, camphorsulfonic acid, ethane sulfonic acid, HCl, HBr, HClO₄, HNO₃, or combinations thereof.

19. The method of any one of claims 1 to 18, wherein the anhydride of step (iv) comprises acetic anhydride, trifluoroacetic acid anhydride, butyric anhydride, propionic anhydride, triflic anhydride, succinic anhydride, maleic anhydride, or combinations thereof.

20. The method of claim 19, wherein the anhydride of step (iv) comprises acetic anhydride, trifluoroacetic, or combinations thereof.

21. The method of any one of claims 1 to 20, wherein the anhydride concentration is from about 0.01% to about 60% by volume of solvent, or from about 0.3% to about 55% by volume of solvent, or from about 1% to about 55% by volume of solvent, or from about 2% to about 50% by volume of solvent; or from about 1% to about 45% by volume of solvent, or from about 2% to about 40% by volume of solvent.

22. The method of any one of claims 1 to 21, wherein:
each R^{1A} and R^{1D} is independently selected from the group consisting of —O—(CH₂)ₙ₁S(O)ᵥ₁X¹, —O—(CH₂)ₙ₁CO₂X¹, and —O—(CH₂)ₙ₁POᵥ₁X¹;
R^{1B} and R^{1C} are hydrogen; and
X¹ is H or alkali metal cation.

23. The method of any one of claims 1 to 22, wherein:
each R^{3A} and R^{3B} is independently C₁-C₃ alkyl, and
R^{4A} and R^{4B} are hydrogen.

24. A method for the preparation of a compound having the formula IV: 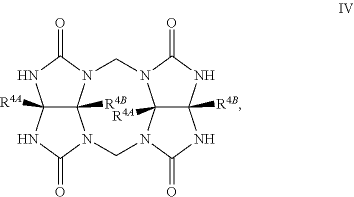
the method comprising:
(i) contacting a compound having the formula II′ 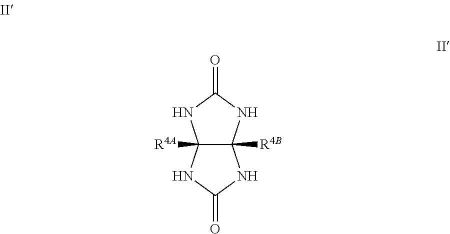
with paraformaldehyde and acid, wherein the paraformaldehyde is added to the compound of formula II′ over a period of at least about 1 hour wherein the reaction is maintained at a temperature above 55° C.; and
(ii) optionally isolating the compound of formula IV.

25. The method of claim 24, wherein the paraformaldehyde is added in four or more portions separated by at least about 10 minutes.

26. The method of claim 25, wherein the paraformaldehyde is added in four or more portions separated by at least about 15 minutes, or by at least about 20 minutes, or by at least about 25 minutes, or by at least about 30 minutes.

27. The method of claim 26, wherein the paraformaldehyde is added in six or more portions separated by at least about 30 minutes.

28. The method of claim 24, wherein in the paraformaldehyde is added by continuous or dropwise addition over a period of at least about 45 minutes, or at least about 1 hour, at least about 1.5 hours, at least about 2 hours, at least about 2.5 hours, at least about 3 hours, at least about 3.5 hours, at least about 4 hours, at least about 4.5 hours, or at least about 5 hours.

29. The method according to any one of claims 24 to 29, wherein the reaction is maintained at a temperature above 65° C.

30. The method according to any one of claims 24 to 29, wherein the reaction mixture does not solidify during the reaction process.

31. The method according to any one of claims 24 to 30, wherein isolation of the compound of formula IV comprises (i) contacting the reaction mixture with water, (ii) heating to a temperature of at least about 70° C., (iii) cooling to a temperature below about 30° C., and (iv) centrifuging or filtering the reaction mixture.

32. The method of any one of claims 24 to 31, wherein R^{4A} and R^{4B} are hydrogen.

33. A method for the preparation of a compound having the formula I 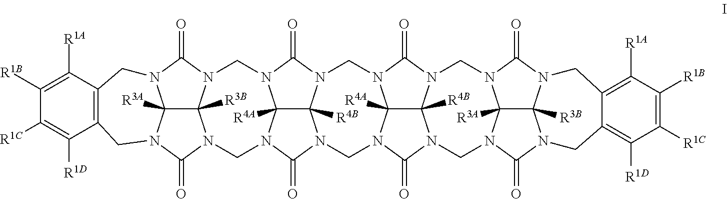
wherein:
each R^{1A} and R^{1D} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹;
each R^{1B} and R^{1C} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹; or
additionally or alternatively, two R^{1A}, R^{1B}, R^{1C} and R^{1D} attached on the same phenyl ring at adjacent positions, together with atoms to which they are attached, are joined to form a fused C₆-C₁₂ aryl, 5 to 12 membered heteroaryl, or 5 to 7 membered heterocycle, which are optionally substituted with 1 to 3 substituents independently selected halogen, —OH, —NH₂, substituted or unsubstituted C₁-C₆ alkyl, or substituted or unsubstituted 2 to 6 membered heteroalkyl;
each R^{3A} and R^{3B} is independently selected from hydrogen, halogen, —OH, C₁-C₆ alkyl, phenyl, substituted phenyl and 2 to 6 membered heteroalkyl;
each R^{4A} and R^{4B} is independently selected from hydrogen, halogen, —OH, C₁-C₆ alkyl, phenyl, substituted phenyl and 2 to 6 membered heteroalkyl;
each n1 is independently selected from 0 to 5;
each v1 is independently selected from 2 or 3; and
each X¹ is independently selected from selected from H, —OH, C₁-C₆ alkyl, alkali metal cation, and quaternary ammonium cation;
the method comprising:
(i) contacting a compound having the formula II′ 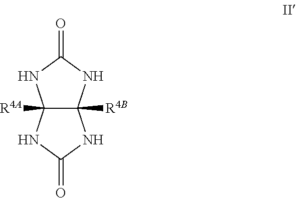
with paraformaldehyde and acid, wherein the paraformaldehyde is added to the compound of formula III over a period of at least about 1 hour, wherein the reaction is maintained at a temperature above 55° C., to provide a compound having the formula IV: 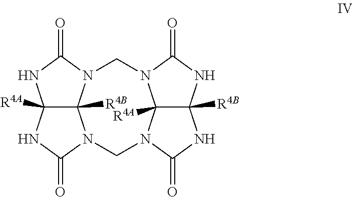
(ii) optionally isolating the compound of formula IV; and
(iii) converting the compound of formula IV to the compound of formula I.

34. The method of claim 33, wherein the paraformaldehyde is added in four or more portions separated by at least about 10 minutes.

35. The method of claim 34, wherein the paraformaldehyde is added in four or more portions separated by at least about 15 minutes, or by at least about 20 minutes, or by at least about 25 minutes, or by at least about 30 minutes.

36. The method of claim 35, wherein the paraformaldehyde is added in six or more portions separated by at least about 30 minutes.

37. The method of claim 33, wherein in the paraformaldehyde is added by continuous or dropwise addition over a period of at least about 45 minutes, or at least about 1 hour, at least about 1.5 hours, at least about 2 hours, at least about 2.5 hours, at least about 3 hours, at least about 3.5 hours, at least about 4 hours, at least about 4.5 hours, or at least about 5 hours.

38. The method according to any one of claims 33 to 37, wherein the reaction is maintained at a temperature above 65° C.

39. The method according to any one of claims 33 to 38, wherein the reaction mixture does not solidify during the reaction process.

40. The method according to any one of claims 33 to 39, wherein isolation of the compound of formula IV comprises (i) contacting the reaction mixture with water, (ii) heating to a temperature of at least about 70° C., (iii) cooling to a temperature below about 30° C., and (iv) centrifuging or filtering the reaction mixture.

41. The method of any one of claims 33 to 40, wherein:
each R^{1A} and R^{1D} is independently selected from the group consisting of —O—(CH₂)ₙ₁S(O)ᵥ₁X¹, —O—(CH₂)ₙ₁CO₂X¹, and —O—(CH₂)ₙ₁POᵥ₁X¹;
R^{1B} and R^{1C} are hydrogen; and
X¹ is H, or alkali metal cation.

42. The method of any one of claims 33 to 41, wherein:
each R^{3A} and R^{3B} is independently C₁-C₃ alkyl, and
R^{4A} and R^{4B} are hydrogen.

43. A method for the preparation of a compound of Formula V: 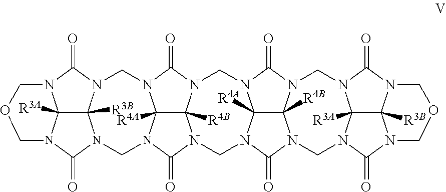
each R^{3A}, R^{3B}, R^{4A}, and R^{4B} is independently hydrogen, halogen, —OH, C₁-C₆ alkyl, 2 to 6 membered heteroalkyl,
the method comprising:
(i) contacting a compound having the formula IV 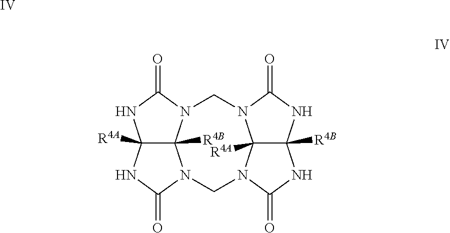
with a compound having the formula III 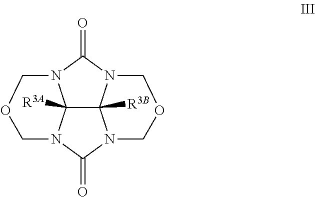
in the presence of (a) one or more acids having a pKa of less than about 1, and optionally (b) one or more polar aprotic solvents and/or additional acid solvents; and
(ii) isolating the compound of formula V.

44. The method of claim 43, wherein the reaction temperature is maintained at or above about 55° C.

45. The method of claim 43 or 44, wherein the acid is selected from the group consisting of trifluoroacetic acid, phosphoric acid, sulfuric acid, HCl, HBr, HClO₄, HNO₃, triflic acid, or combinations thereof.

46. The method according to any one of claims 43 to 45, wherein the solvent comprises an acid selected from the group consisting of trifluoroacetic acid, phosphoric acid, sulfuric acid, HCl, HBr, HClO₄, HNO₃, triflic acid, methane sulfonic acid, toluene sulfonic acid, Eaton's reagent, and combinations thereof.

47. The method according to any one of claims 43 to 46, wherein the polar aprotic solvent comprises DMF, DMSO, acetonitrile, DMA, NMP, THF, dioxane, sulfolane, acetone, DMPU, diglymes, HMPA, or combinations thereof.

48. The method according to any one of claims 43 to 47, which comprises a solvent which includes trifluoroacetic acid.

49. The method according to any one of claims 43 to 48, wherein the method further comprises:
(ii) adding additional TFA to the reaction mixture after completion of the reaction;
(iii) precipitating the compound of formula V; and
(iv) filtering a mixture comprising the compound of formula V.

50. The method of claim 49, wherein the precipitation of the compound of formula V comprises the addition of a countersolvent for the compound of formula V, and the countersolvent comprises ethanol, brine, MeOH, acetone, iPA, THF, or combinations thereof.

51. The method of any one of claims 43 to 50, wherein:
each R^{3A} and R^{3B} is independently C₁-C₃ alkyl, and
R^{4A} and R^{4B} are hydrogen.

52. A method for the preparation of a compound having the formula I 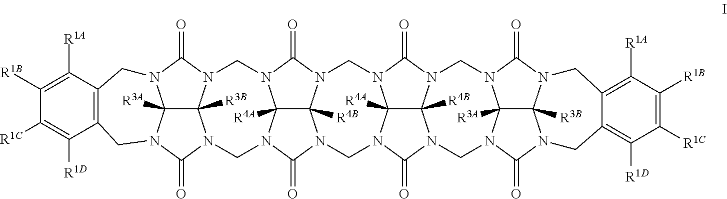
wherein:
each R^{1A} and R^{1D} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹;
each R^{1B} and R^{1C} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹; or
additionally or alternatively, two R^{1A}, R^{1B}, R^{1C} and R^{1D} attached on the same phenyl ring at adjacent positions, together with atoms to which they are attached, are joined to form a fused C₆-C₁₂ aryl, 5 to 12 membered heteroaryl, or 5 to 7 membered heterocycle, which are optionally substituted with 1 to 3 substituents independently selected halogen, —OH, —NH₂, substituted or unsubstituted C₁-C₆ alkyl, or substituted or unsubstituted 2 to 6 membered heteroalkyl;
each R^{3A} and R^{3B} is independently selected from hydrogen, halogen, —OH, C₁-C₆ alkyl, phenyl, substituted phenyl and 2 to 6 membered heteroalkyl;
each R^{4A} and R^{4B} is independently selected from hydrogen, halogen, —OH, C₁-C₆ alkyl, phenyl, substituted phenyl and 2 to 6 membered heteroalkyl;
each n1 is independently selected from 0 to 5;
each v1 is independently selected from 2 or 3; and
each X¹ is independently selected from selected from H, —OH, C₁-C₆ alkyl, alkali metal cation, and quaternary ammonium cation;
the method comprising:
(i) preparing an intermediate having the formula V 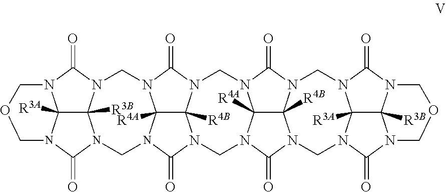
comprising contacting a compound having the formula IV 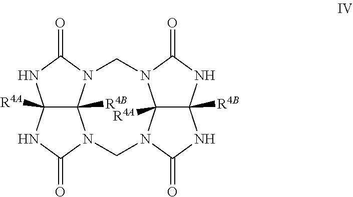
and a compound having the formula III 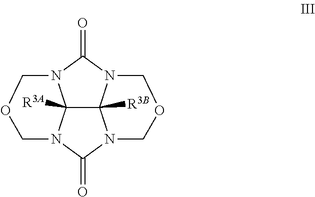
in the presence of (a) one or more acids having a pKa of less than about 1, and optionally (b) one or more polar aprotic solvents and/or additional acid solvents;
(ii) isolating the compound of formula V; and
(iii) converting the compound of formula V to the compound of formula I.

53. The method of claim 52, wherein each R^{1A} and R^{1D} is —O—(CH₂)₃—SO₃Na.

54. The method of claim 52 or 53, wherein the reaction temperature of step (i) is maintained at or above about 55° C.

55. The method of claim 52 or 54, wherein the acid is selected from the group consisting of trifluoroacetic acid, phosphoric acid, sulfuric acid, HCl, HBr, HClO₄, HNO₃, triflic acid, or combinations thereof.

56. The method according to any one of claims 52 to 55, wherein the solvent comprises an acid selected from the group consisting of trifluoroacetic acid, phosphoric acid, sulfuric acid, HCl, HBr, HClO₄, HNO₃, triflic acid, methane sulfonic acid, toluene sulfonic acid, Eaton's reagent, and combinations thereof.

57. The method according to any one of claims 52 to 56, wherein the polar aprotic solvent comprises DMF, DMSO, acetonitrile, DMA, NMP, THF, dioxane, sulfolane, acetone, DMPU, diglymes, HMPA, or combinations thereof.

58. The method according to any one of claims 52 to 57, which comprises a solvent which includes trifluoroacetic acid.

59. The method according to any one of claims 52 to 58, which further comprises adding TFA to the reaction mixture after completion of the reaction, precipitating the compound of formula V, and isolating the compound of formula V comprising filtration.

60. The method of claim 59, wherein the precipitation of the compound of formula V comprises the addition of a countersolvent for the compound of formula V, which is selected from the group consisting of ethanol, brine, MeOH, acetone, iPA, THF, or combinations thereof.

61. The method of any one of claims 52 to 60, wherein:
each R^{1A} and R^{1D} is independently selected from the group consisting of —O—(CH₂)ₙ₁S(O)ᵥ₁X¹, —O—(CH₂)ₙ₁CO₂X¹, and —O—(CH₂)ₙ₁POᵥ₁X¹;
R^{1B} and R^{1C} are hydrogen; and
X¹ is H, or alkali metal cation.

62. The method of any one of claims 52 to 61, wherein:
each R^{3A} and R^{3B} is independently C₁-C₃ alkyl, and
R^{4A} and R^{4B} are hydrogen.

63. A method for the preparation of a compound of Formula VI-A: 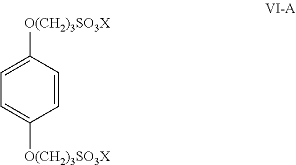
wherein:
X is selected from H and alkali metal cation;
the method comprising:
(i) contacting a hydroquinone having the formula 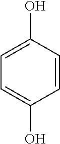
and a sultone having the formula 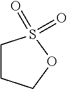
in the presence of about 2.0 to 2.5 equivalents of a base, and a solvent comprising one or more of water, polar aprotic solvents, and combinations thereof.

64. The method of claim 63, wherein the base is present at about 2.2 to 2.4 equivalents.

65. The method of claim 63 or 64, wherein the base comprises an alkali metal hydroxide.

66. The method according to any one of claims 63 to 65, wherein the sultone is added over a period of greater than about 2 hours.

67. The method according to claim 66, wherein the sultone is added in two or more portions separated by at least one hour.

68. The method according to any one of claims 63 to 67, wherein the reaction mixture is cooled to less than about 10° C. prior to each addition of the sultone.

69. The method according to any one of claims 63 to 68, further comprising adding a base to the reaction in an amount equivalent to or greater than an amount of the sultone.

70. The method according to claim 69, wherein the base is added in two or more portions separated by at least one hour.

71. A method for the preparation of a compound having the formula I-A 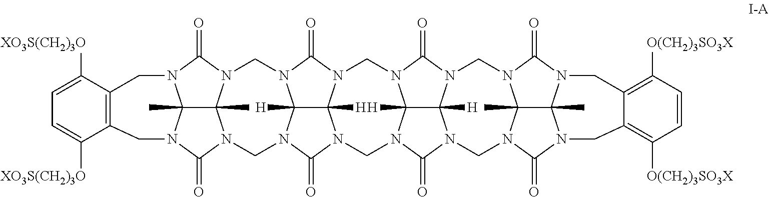
wherein
X is selected from H and alkali metal cation;
the method comprising:
(i) contacting a hydroquinone having the formula 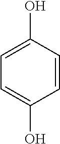
and a sultone having the formula 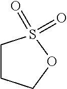
in the presence of about 2.0 to 2.5 equivalents of a base, and a solvent comprising one or more of water, polar aprotic solvents, and combinations thereof, to provide a compound having formula VI-A: 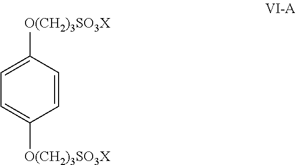
(ii) isolating the compound of formula VI-A; and
(iii) contacting the compound of formula VI-A and a compound having the formula V-A 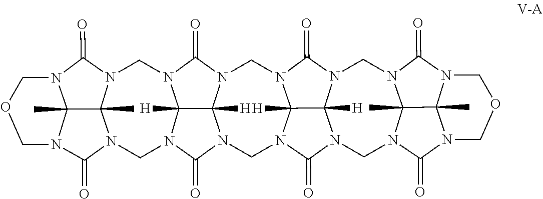
to provide the compound having the formula I-A.

72. The method of claim 71, wherein the base of step (i) is present at about 2.2 to 2.4 equivalents.

73. The method of claim 71 or 72, wherein the base of step (i) comprises an alkali metal hydroxide.

74. The method according to any one of claims 71 to 73, wherein the sultone of step (i) is added over a period of greater than about 2 hours.

75. The method according to claim 74, wherein the sultone is added in two or more portions separated by at least one hour.

76. The method according to any one of claims 71 to 75, wherein the reaction mixture of step (i) is cooled to less than about 10° C. prior to each addition of the sultone.

77. The method according to any one of claims 71 to 76, further comprising adding a base to the reaction in an amount equivalent to or greater than an amount of the sultone.

78. The method according to claim 77, wherein the base is added in two or more portions separated by at least one hour.

79. A method for the preparation of a compound having the formula I 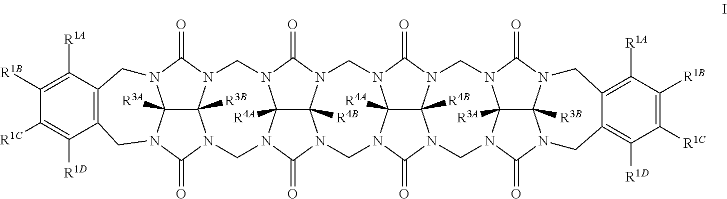
wherein:
each R^{1A} and R^{1D} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹;
each R^{1B} and R^{1C} is independently selected from hydrogen, halogen, —OH, C₁-C₆ alkyl, 2 to 6 membered heteroalkyl, C₃-C₆ cycloalkyl, 5 to 6 membered heterocycloalkyl, phenyl, 5 to 6 membered heteroaryl, —O—(CH₂)ₙ₁S(O)ᵥ₁X¹, —O—(CH₂)ₙ₁CO₂X¹, and —O—(CH₂)ₙ₁POᵥ₁X¹; or
additionally or alternatively, two R^{1A}, R^{1B}, R^{1C} and R^{1D} attached on the same phenyl ring at adjacent positions, together with atoms to which they are attached, are joined to form a fused C₆-C₁₂ aryl, 5 to 12 membered heteroaryl, or 5 to 7 membered heterocycle, which are optionally substituted with 1 to 3 substituents independently selected halogen, —OH, —NH₂, substituted or unsubstituted C₁-C₆ alkyl, or substituted or unsubstituted 2 to 6 membered heteroalkyl;
each R^{3A} and R^{3B} is independently selected from hydrogen, halogen, —OH, C₁-C₆ alkyl, phenyl, substituted phenyl and 2 to 6 membered heteroalkyl;
each R^{4A} and R^{4B} is independently selected from hydrogen, halogen, —OH, C₁-C₆ alkyl, phenyl, substituted phenyl and 2 to 6 membered heteroalkyl;
each n1 is independently selected from 0 to 5;
each v1 is independently selected from 2 or 3; and
each X¹ is independently selected from selected from H, —OH, C₁-C₆ alkyl, alkali metal cation, and quaternary ammonium cation;
the method comprising:
contacting the compound having the formula V 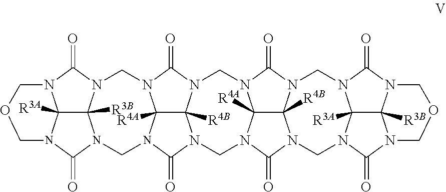
and a compound having the formula VI: 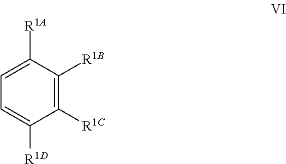
in the presence of a solvent comprising one or more acids having a pKa of less than about 1 and anhydride to provide the compound having the formula I.

80. The method of claim 79, wherein:
each R^{1A} and R^{1D} is independently selected from the group consisting of —O—(CH₂)ₙ₁S(O)ᵥ₁X¹, —O—(CH₂)ₙ₁CO₂X¹, and —O—(CH₂)ₙ₁POᵥ₁X¹;
R^{1B} and R^{1C} are hydrogen; and
X¹ is H, or alkali metal cation.

81. The method of claim 79 or 80, wherein:
each R^{3A} and R^{3B} is independently C₁-C₃ alkyl, and
R^{4A} and R^{4B} are hydrogen.

82. The method of claim 79 or 81, wherein:
each R^{3A} and R^{3B} are methyl,
R^{4A} and R^{4B} are hydrogen, and
R^{1A} and R^{1D} are —O—(CH₂)ₙ₁S(O)ᵥ₁X¹.
each n1 is 3;
each v1 is 3; and
each X¹ is independently selected from selected from H or alkali metal cation.

83. The method according to any one of claims 79 to 82, wherein the anhydride is selected from a compound having the structure 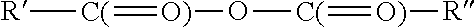
wherein R′ and R″ may me the same or different and are independently selected from alkyl, aryl, heteroalkyl, or may be taken together to form a heterocyclic ring, each of which may be optionally substituted by one or more halo, alkyl or trihaloalkyl.

84. The method according to any one of claims 79 to 82, wherein the anhydride is selected from acetic anhydride, trifluoroacetic acid anhydride, butyric anhydride, propionic anhydride, triflic anhydride, succinic anhydride, maleic anhydride, or combinations thereof.

85. The method according to any one of claims 79 to 82, wherein the anhydride is selected from acetic anhydride, trifluoroacetic anhydride, or a combination thereof.

86. The method of any one of claims 79 to 85, wherein the anhydride concentration is from about 0.01% to about 60% by volume of solvent, or from about 0.3% to about 55% by volume of solvent, or from about 1% to about 55% by volume of solvent, or from about 2% to about 50% by volume of solvent; or from about 1% to about 45% by volume of solvent, or from about 2% to about 40% by volume of solvent.

87. The method according to any one of claims 79 to 86, wherein the reaction employs about 4 equivalents or less of compound of Formula VI, or about 3 equivalents or less of compound of Formula VI.

88. The method according to any one of claims 79 to 86, wherein the reaction employs from about 2 equivalents to about 5 equivalents of compound of Formula VI, or from about 2.5 equivalents to about 4.5 equivalents of compound of Formula VI, or from about 2.5 equivalents to about 4 equivalents of compound of Formula VI.
